(19) United States
(12) Patent Application Publication
(10) Pub. No.: US 2024/0299528 A1
(43) Pub. Date: Sep. 12, 2024

(54) A DNA PLASMID SARS-CORONA VIRUS-2/COVID-19 VACCINE

(71) Applicant: STATENS SERUM INSTITUT, Copenhagen S (DK)

(72) Inventors: Charlotta Polacek STRANDH, Copehagen S (DK); Anders FOMSGAARD, Copehagen S (DK)

(21) Appl. No.: 18/004,148

(22) PCT Filed: Jul. 2, 2021
(86) PCT No.: PCT/EP2021/068324

§ 371 Date: Jan. 3, 2023

(30) Foreign Application Priority Data
Jul. 3, 2020 (EP) 20183972.7
Sep. 30, 2020 (EP) 20199341.7

(51) Int. Cl. A61K 39/215; A61K 39/00; A61P 37/04; C07K 14/005

(52) U.S. Cl. CPC A61K 39/215; A61P 37/04; C07K 14/005; A61K 2039/53; A61K 2039/54; A61K 2039/545; C12N 2770/20022; C12N 2770/20034; C12N 2770/20071

(57) ABSTRACT

The present invention relates to DNA vaccine against SARS-Coronavirus-2 (SARS-CoV-2) infection. In particular, the present invention relates to a DNA vaccine encoding the SARS-Coronavirus-2 spike protein for use in prevention or treatment of viral infection in humans and/or animals.

The DNA vaccine including the DNA construct has several features in its design that together provide a more safe and broad protection against SARS-Cov-2 strains in humans and animals, e.g. mink, ferrets, pigs and cats. The DNA construct encodes the SPIKE protein derived from the pandemic strain; Wuhan-Hu-1 (MN908947). The sequence is codon optimized for high expression in human and mammalian cells and the DNA construct is inserted in a selected DNA plasmid for eukaryotic in vivo and in vitro expression. The combination of the choice of SARS-COV-2 SPIKE sequence, codon optimization, expression in the new generation eukaryotic expression plasmid with no antibiotic resistance marker (instead the RNA-OUT system is used for safety) and delivery to the very immunogenic skin, results in protection against SARS-COV-2 infection and covid-19 disease.

### CROSS-REFERENCE OF RELATED APPLICATIONS

This application is a § 371 of International Application No. PCT/EP2021/068324, filed Jul. 2, 2021, which claims the benefit of priority to EP Provisional Application Nos. 20/183,972.7, filed Jul. 3, 2020, and 20199341.7, filed Sep. 30, 2020, each of which are incorporated by reference in their entirety.

### TECHNICAL FIELD OF THE INVENTION

The present invention relates to DNA vaccine against SARS-Coronavirus-2 infection. In particular, the present invention relates to a DNA vaccine encoding the SARS-Coronavirus-2 SPIKE protein for use in prevention or treatment of viral infection in humans and animals.

### Incorporation-by-Reference of Material Submitted in Electronic Form

Incorporated herein by reference in its entirety is the sequence listing submitted via EFS-Web as a text file named SSI-27-US_20230714_SEQLIST_ST25.txt, created Jul. 14, 2023, and having a size of 74,483 bytes.

### BACKGROUND OF THE INVENTION

The world is in the middle of a global health emergency, the need for efficient and easy to produce vaccines has never been more relevant.

The severe acute respiratory syndrome coronavirus-2 (SARS-COV-2), is a novel human pathogen that emerged in Wuhan, China, in December 2019 (www.who.int/emergencies/diseases/novel-coronavirus-2019). It causes a severe lung infection disease named Covid-19. It has since spread to >215 countries and territories, causing a global pandemic. As of 6Sep. 2020, over 26 million confirmed cases and 850.000 deaths has been reported worldwide (WHO COVID-19 Weekly Epidemiological Update; 6 Sep. 2020). The associated disease, coronavirus disease 2019 (COVID-19), is characterized by a dry cough, fever, and fatigue. While the majority of infected individuals will experience mild-to-moderate symptoms, approximately 4.6 in 100 000 will require hospitalization (Garg et al 2020), one third of whom will develop respiratory failure and require mechanical ventilation (Goyal et al 2020). Other complications include multiorgan failure and death.

The groups at risk of severe disease are individuals older than 65 years of age and those with underlying conditions that include hypertension, obesity, chronic lung disease, diabetes mellitus, and cardiovascular disease (Garg et al 2020). There is currently no effective prophylaxis or treatment.

In the majority of countries, the epidemic has not yet reached its peak and it is predicted that the number of cases and deaths will continue to rise in the coming weeks and months. While social distancing, improved hygiene and country-wide lockdowns have successfully slowed the spread in some places, immune naïve individuals remain at risk of developing severe disease and spreading the virus, especially once restrictions are lifted. Inducing immunity through vaccination of the naïve group may be an effective mean of preventing disease and sequelae, minimizing further spread, protecting risk groups and alleviating further strain on the health care systems worldwide. It will also enable opening of societies and restarting failing economies. In addition, as new SARS-COV-2 variants emerge, there is a risk that the acquired immunity through vaccination or infection could erode. It may therefore be beneficial to vaccinate naïve groups, or boost already vaccinated/recovered individuals, with updated version of the vaccines. There are a few known amino acid substitutions in SPIKE that reduce neutralization by antibodies raised against the wild type SARS-COV-2. Two such positions are E484K and K417N, present in SPIKE of e.g. the SARS-COV-2 variant of concern B.1.351, which may therefore be well suited as a template for the next generation of SARS-COV-2 vaccines by providing an alternative or broadened immunity.

Vaccination is a preferred choice for Covid-19 prophylaxis. Active immunization with viral antigens can be achieved through different vaccine platforms, these include live attenuated, inactivated, subunit protein, viral vectored, and plasmid DNA or RNA vaccines. Due to the intrinsic nature of naked plasmid DNA vaccines as potent inducers of cellular immune responses (Th1 responses and broader antibody responses), DNA vaccines have the potential to not only elicit neutralizing antibody responses to block infection (sterilizing immunity), but also to limit disease severity of breakthrough infections through cellular immunity and antibody dependent cell cytotoxicity (ADCC). Historically, naked plasmid DNA vaccines have had excellent safety profiles and are Generally Regarded As Safe (GRAS) vaccines. Thus, for many plasmid DNA vaccines not even toxicity tests in animals are required.

During the Covid-19 pandemic, some clinical studies have already been initiated using either protein, recombinant virus, RNA vaccine and DNA plasmid vaccines, so far with no reported serious adverse events (SAE).

Although plasmid DNA vaccines were developed more than 25 years ago, clinical trials preceding stage I and II in humans are rare. Currently, about one hundred stage I and II clinical trials for DNA vaccines in humans are being conducted (Rosa, 2015). However, three prophylactic veterinary DNA vaccines have been licensed: one for West Nile Virus (in horses) and a second for Infectious Hematopoietic Necrosis virus in Salmon, and an immunotherapeutic vaccine for cancer in dogs (Liu 2011). A forth DNA plasmid construct is licensed as a growth hormone therapy for pigs (production animals) (Liu 2011). This demonstrates that DNA vaccines can have good and protective effects and that new DNA vaccines are not limited by the size of the animal or species (Kutzler 2008). The great success of DNA vaccines, observed for the murine model with the first generation of DNA vaccines, did initially not translate well into humans. However, the field has moved significantly forward through improvements of gene expression, the vaccine gene constructs, the vector backbones, use of adjuvants, the delivery methods, the vaccine modality such as different prime-boost strategies, DNA dose and vaccine intervals, and have together made the nucleotide vaccines highly clinically relevant (Liu 2011, Kutzler 2008, Jones 2009). Researchers have recently demonstrated protective antibodies levels by a single dose of gene gun administrated influenza A virus hemagglutinin (HA) DNA vaccine to humans.

Although “Nucleic acid immunization”, which is sometimes used instead of the commonly used term “DNA vaccines”, could cover both naked DNA and RNA, and perhaps even recombinant virus-vector delivery, we use the term DNA vaccine for naked circular plasmid DNA. DNA vaccination is the inoculation of antigen-encoding plasmid DNA derived from wild type or synthetic sequence origin, incorporated into expression cassette or plasmid vector in order to induce immunity to the encoded antigen. The vaccine sequence of interest, encoding the SARS-COV-2 SPIKE, is incorporated in a naked circular plasmid with the key features necessary for expression from DNA and production (e.g. origin of replication, promotor, sequence of interest and polyadenylation signal).

Delivery systems may most often be naked plasmid DNA in buffer with or without adjuvant (WO2016041562), DNA coupled to nanoparticles and/or formulated into adjuvant containing compounds (Liu 2011).

More than 10 years ago, the SARS-COV-2-related corona virus SARS-COV-1 spread around the globe and led to the first pandemic of the 21^{st} century. DNA vaccines against SARS-COV-1, have been proposed in the fight against SARS-CoV-2, but as very little similarity among the corona viruses exist, the change of cross protection when transferring the vaccine from one virus to another might not be expected.

WO2005021707 describes a DNA vaccine directed against the SPIKE protein of the previously circulating SARS-COV-1, which is a protein located on the outside of the virus helping the virus to enter the host cell of an infected subject. A SPIKE protein is present on the surface of SARS-Cov-2 as well, but as it only shares about 76% amino acid sequence identity with SPIKE from SARS-COV-1, a vaccine designed for SPIKE on SARS-COV-1 is less likely to protect against SARS-COV-2.

Hence, a vaccine directed to stimulate a strong immune response against the SARS-COV-2 would be advantageous, and in particular a vaccine that is directed to stimulate both humoral as well as cell-mediated immunity.

### SUMMARY OF THE INVENTION

Thus, an object of the present invention relates to the provision of a DNA vaccine for use in prevention or treatment of viral infections.

In particular, it is an object of the present invention to provide a nucleotide SARS-CoV-2 vaccine that solves the above mentioned problems of the prior art.

Thus, one aspect of the invention relates to a DNA vaccine comprising a DNA construct with the nucleic acid sequence SEQ ID NO: 1 encoding a modified SPIKE protein that originates from the corona virus SARS-COV-2 and/or the nucleic acid sequence SEQ ID NO: 12 encoding a modified SPIKE protein that originates from SARS-COV-2 variant B.1.351 or a fragment thereof having at least 80% sequence identity to SEQ ID NO: 1 or 12, preferably 90%, more preferably 95% sequence identity to SEQ ID NO: 1 or 12.

Another aspect of the present invention relates to a DNA vaccine comprising a DNA construct with the nucleic acid sequence SEQ ID NO: 2 encoding a modified SPIKE S1 protein that originates from the corona virus SARS-COV-2 and/or the nucleic acid sequence SEQ ID NO: 13 encoding a modified SPIKE S1 protein that originates from SARS-COV-2 variant B.1.351 or a fragment thereof having at least 80% sequence identity to SEQ ID NO: 2 or 13, preferably 90%, more preferably 95% sequence identity to SEQ ID NO: 2 or 13.

Yet another aspect of the present invention is to provide a DNA vaccine comprising a DNA construct with the nucleic acid sequence SEQ ID NO: 3 encoding a modified SPIKE S2 protein that originates from the corona virus SARS-CoV-2 and/or the nucleic acid sequence SEQ ID NO: 14 encoding a modified SPIKE S2 protein that originates from SARS-COV-2 variant B.1.351 or a fragment thereof having at least 80% sequence identity to SEQ ID NO: 3 or 14, preferably 90%, more preferably 95% sequence identity to SEQ ID NO: 3 or 14.

Still another aspect of the present invention is to provide a DNA vaccine comprising a DNA construct with the nucleic acid sequence SEQ ID NO: 4 encoding a modified receptor binding motif (RBM) protein that originates from the corona virus SARS-COV-2 and/or the nucleic acid sequence SEQ ID NO: 15 encoding a modified receptor binding motif (RBM) protein that originates from SARS-COV-2 variant B.1.351 or a fragment thereof having at least 80% sequence identity to SEQ ID NO: 4 or 15, preferably 90%, more preferably 95% sequence identity to SEQ ID NO: 4 or 15.

A further aspect of the present invention is to provide a DNA vaccine comprising a DNA construct with the nucleic acid sequence SEQ ID NO: 5 encoding a modified receptor binding domain (RBD) protein that originates from the corona virus SARS-COV-2 and/or the nucleic acid sequence SEQ ID NO: 16 encoding a modified receptor binding domain (RBD) protein that originates from SARS-COV-2 variant B.1.351 or a fragment thereof having at least 80% sequence identity to SEQ ID NO: 5 or 16, preferably 90%, more preferably 95% sequence identity to SEQ ID NO: 5 or 16.

An even further aspect of the present invention is to provide a DNA vaccine comprising a DNA construct encoding an amino acid sequence according to SEQ ID NO: 6 encoding a modified SPIKE protein that originates from the corona virus SARS-COV-2 and/or a DNA construct encoding an amino acid sequence according to SEQ ID NO: 17 encoding a modified SPIKE protein that originates from the corona virus SARS-COV-2 variant B.1.351 or a fragment thereof having at least 80% sequence identity to SEQ ID NO: 6 or 17, preferably 90%, more preferably 95% sequence identity to SEQ ID NO: 6 or 17.

Yet a further aspect of the present invention is to provide a pharmaceutical composition comprising a DNA construct inserted in an expression vector as described herein.

Still a further aspect of the present invention is the use of a DNA vaccine as described for preparation of a medicament for inducing a protective immune response against SARS-COV-2

### BRIEF DESCRIPTION OF THE FIGURES

FIG. 1 shows specific antibody responses in mice vaccinated with SARS-COV-2 SPIKE DNA vaccine. Antibody titers in serum obtained from mice vaccinated intradermal on day 0, 10 and 26 with 50 or 17 μg SARS-COV-2 DNA vaccine, respectively, or 50 μg of non-coronavirus DNA vector were determined using ELISA. A. Antibody titers obtained against complete SARS-COV-2 SPIKE protein. B. Antibody titers obtained against the receptor binding domain (RBD) of SARS-CoV-2 SPIKE protein. n=5 mice per group. Error bars represent the SEM.

FIG. 2 shows neutralizing antibodies induced by SARS-COV-2 SPIKE DNA vaccine. Neutralizing antibody titers in serum obtained on day 40 from mice vaccinated intradermal at day 0, 10 and 26 with 50 or 17 μg SARS-COV-2 DNA vaccine, respectively, or 50 μg of non-coronavirus DNA vector (n=5 mice per group). Human SARS-COV-2 convalescence plasma were used as a titer reference to determine functional range of neutralization.

Neutralization titers were determined in a microneutralization test using a Danish SARS-COV-2 isolate.

FIG. 3 shows cellular immune response induced by SARS-COV-2 DNA vaccine in mice. Mice were immunized three times (day 0, 10 and 26) with SARS-COV-2 SPIKE encoding DNA vaccine and spleens were harvested on day 40. Cellular immune response was measured by re-stimulation of the spleenocytes with SARS-CoV-2 SPIKE, SARS-COV-2 RBD, hCoV-HKU1 SPIKE, hCoV-229E SPIKE, PBS (negative control) or Concanavalin A (positive control), respectively, and measuring the cytokine production of A) INF-gamma (Th1 response), B) IL-5 (Th2 response) and C) IL-17a (Th17 response) with cytokine-specific ELISAs.

FIG. 4 shows the experimental setup for testing of the SARS-COV-2 DNA vaccine on non-human primates rhesus macaques. Seven Rhesus macaques (2 to 8 years old) were divided into two groups, wherein five received the SARS-COV-2 DNA vaccine and two received a sham control. The animals received three immunizations of 2 mg DNA without adjuvant by intradermal route at week 0, 2 and 4.

FIG. 5 shows the immune response induced by the SARS-COV-2 DNA vaccine in non-human primates. FIG. 5a shows the levels of SPIKE-specific binding antibodies in the animals after immunization. FIG. 5b shows the evaluation of neutralizing antibody responses using a live virus plaque reduction neutralization test (PRNT).

FIG. 6 shows the protective efficacy of the SARS-COV-2 DNA vaccine. Non-human primates were challenged 4 weeks after final immunization and virus was measured in bronchoalveolar lavage (BAL) and nasal swabs. FIG. 6a shows peak viral load in SARS-COV-2 DNA vaccinated and sham animals. FIG. 6b shows replicating virus in BAL and nasal swabs, post challenge of vaccinated and sham animals. Bold line indicates median responses at each time point.

FIG. 7 shows the immunogenicity of the Wuhan-like SARS-COV-2 DNA vaccine in comparision to a B.1.351-variant based SARS-COV-2 DNA vaccine (SPIKE B.1.351 vaccine. Rabbits were immunized with the two different DNA vaccines, as outlined in FIG. 7A. Antibody levels and broadness of immunity was analyzes by IgG ELISA, specific for four different SPIKE variants or four different RBD variants (FIG. 7B). The neutralizing properties of the elicited antibodies were evaluated by a live virus neutralization test in an ELISA format and 50% virus neutralization titers were calculated (FIG. 7C). The cell mediated immunity was measured in rabbit splenocytes from animals two weeks after the third vaccination using IFNγ-ELISA (FIG. 7D) and IFNγ-ELISPOT (FIG. 7E). The splenocytes were re-stimulated with various SPIKE- and RBD proteins and excreted IFN-γ from stimulated cells were measured as an indicator of cell mediated immunity. A non-related protein (Influenza HA protein) and cell culture media were used as a non-specific control. White arrows indicate homologous response.

FIG. 8 shows the booster effect of a heterogenous immunization regiment (outline, FIG. 8A). Antibody levels at two weeks post final immunization were measured using IgG ELISAs specific for four different SPIKE variants and three different RBD variants (FIG. 8B). White arrows indicate homologous response. The antibody levels after the second and third vaccination were calculated as the geometric mean from SPIKE- and RBD-specific ELISA endpoint titers (FIG. 8C).

### DETAILED DESCRIPTION OF THE INVENTION

### Definitions

Prior to discussing the present invention in further details, the following terms and conventions will first be defined:

### Concanavalin A

In the present context, the term “Concanavalin A” is here defined as a lectin (carbohydrate-binding protein). It binds to certain structures found in various sugars, glycoproteins and glycolipids. It can stimulate mouse T cell subsets giving rise to different functionally distant T cell populations.

hCoV-HKU1 SPIKE

In the present context, the term “hCoV-HKU1 SPIKE” referes to the viral SPIKE protein present in the human common cold coronavirus strain HKU1 (a beta-coronavirus, alike SARS-COV-2). hCoV-HKU1 is commonly circulating in the human population.

hCoV-229E SPIKE

In the present context, the term “hCoV-229E SPIKE” referes to the viral SPIKE protein present in the human common cold coronavirus strain 229E (an alpha-coronavirus). hCoV-229E is commonly circulating in the human population.

### Codon Optimization

In the present context, the term “Codon optimization” is here defined as a process used to improve gene expression and increase the translational efficiency of a gene of interest by accommodating codon bias of the host organism.

### SARS-COV-2

In the present context, the term “SARS-COV-2” refers to the virus “Severe Acute Respiratory Syndrome-Corona-Virus-2”, an RNA virus member of the coronavirus family, within the genus betacoronavirus. The virus causes coronavirus disease 2019 (COVID-19), which is a disease affecting the respiratory system of the infected subject.

### SPIKE

In the present context, the term “SPIKE” refers to a homotrimeric glycoprotein comprising the subunits S1 and S2. SPIKE is located on the surface of SARS-COV-2, where it binds the cellular receptor ACE2. Upon binding of SPIKE to its receptor, the virus gets access to its host cell in the infected subject.

### SPIKE B.1.351

In the present context, the term “SPIKE B.1.351” refers to the SPIKE sequence of a recently emerged SARS-COV-2 variant of concern, termed B.1.351 or beta. In the SPIKE coding region, this variant differs in 11 amino acid positions from the reference SARS-COV-2 virus isolated from Wuhan.

### RBD

In the present context, the term “RBD” refers to “Receptor binding domain”, a part of the SPIKE glycoprotein. The region encoding RBD is located in the S1 subunit of the SPIKE gene. RBD is the area of SPIKE, where the ACE2 receptor binds.

### RBM

In the present context, the term “RBM” refers to “Receptor biding motif”, a part of the SPIKE glycoprotein. The gene encoding RBM is located inside RBD part of SPIKE and is the specific area, where the interaction with the ACE2 receptor takes place.

### Epitope

In the present context, the term “epitope” refers to the part of an antigen, which is recognized by the immune system.

### MHC Class I/II Protein

In the present context, the term “MHC class I/II” refers to the two primary classes of major histocompatibility complex molecules. MHC class I are found on the surface of all nucleated cells in the bodies of vertebrates, whereas MHC class II are found only on professional antigen-presenting cells such as dendritic cells, mononuclear phagocytes and B-cells. Their function is to display fragments derived from cytosolic as well as extracellular protein to either cytotoxic T cells or helper T-cell.

### Eukaryotic Expression Vector

In the present context, the term “eukaryotic expression vector” refers to a tool used to introduce a specific coding polynucleotide sequence into a target cell, comprising expression control sequences operatively linked to a nucleotide sequence to be expressed.

### Immunization

In the present context, the term “immunization” refers to the process, whereby a subject is getting immune or resistant to an infection.

### Intradermal

In the present context, the term “intradermal” refers to a way of injecting a substance into the dermis, which is the middle layer of the skin, of a subject.

### Intravenous

In the present context, the term “intravenous” refers to a way of injecting a substance into the veins of a subject.

### Intramuscular

In the present context, the term “intramuscular” refers to a way of injecting a substance into the muscles of a subject.

### Subcutaneous

In the present context, the term “subcutaneous” refers to a way of injecting a substance into the tissue layer situated under the skin of the subject.

### Adjuvants

In the present context, the term “Adjuvants” refers to a compound or mixture that stabilizes the DNA vaccine and/or facilitates transfection of cells with the vaccine or a compound that enhances the immune response to an antigen. An adjuvant can serve as a tissue depot that slowly releases the antigen and as a lymphoid system activator, which non-specifically enhances the immune response. Often, a primary challenge with an antigen alone, in the absence of an adjuvant, will fail to elicit a humoral or cellular immune response.

### Promoter

In the present context, the term “promoter” refers to a sequence of DNA to which proteins binds in order to initiate transcription of DNA into RNA.

### Terminator

In the present context, the term “terminator” refers to a section in a nucleic acid sequence that mediates transcriptional termination of the gene leading to release of the transcribed RNA from the transcriptional complex.

### Subject

The term “subject” comprises humans of all ages, other primates (e.g., cynomolgus monkeys, rhesus monkeys); mammals in general, including commercially relevant mammals, such as cattle, pigs, horses, sheep, goats, mink, ferrets, hamsers, cats and dogs, as well as birds. Preferred subjects are humans.

The term “subject” also includes healthy subjects of the population and, in particular, healthy subjects, who are exposed to pathogenes and in need of protection against infection, such as health personnel.

### Sequence Identity

In the present context, the term “sequence identity” is here defined as the sequence identity between genes or proteins at the nucleotide, base or amino acid level, respectively. Specifically, a DNA and an RNA sequence are considered identical if the transcript of the DNA sequence can be transcribed to the corresponding RNA sequence.

Thus, in the present context, “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical in that position. The percent identity between the two sequences is a function of the number of identical positions shared by the sequences (i.e., % identity=#of identical positions/total #of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the BLASTN and BLASTX programs of (Altschul et al. 1990). BLAST nucleotide searches may be performed with the NBLAST program, to obtain nucleotide sequences homologous to a nucleic acid molecule of the invention. BLAST protein searches may be performed with the BLASTX program, to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilising the BLASTN, BLASTX, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

### Transfection Facilitating Agent/Material/Compound

In the present context, the term “transfection facilitating agent/material/compound” refers to an agent, compound or material that facilitate delivery of polynucleotides to the interior of a cell and/or to a desired location within a cell. It should be noted that certain transfection facilitating materials/agents/compounds might also be “adjuvants” according to the definition as described herein.

Examples of the transfection facilitating compounds include, but are not limited to inorganic materials such as calcium phosphate, aluminium sulfate and gold particles, peptides, proteins, lipids, polymers. A transfection facilitating material can be used alone or in combination with one or more other transfection facilitating materials.

### TCID50

In the present context, the term “TCID50” referes to median tissue culture infectious dose and signifies the concentration at which 50% of the cells are infected when a test tube upon which cells have been cultured, is inoculated with a diluted solution of a viral fluid.

### The Invention Will Now be Described in More Details:

The present invention provides a DNA vaccine comprising a DNA construct comprising a modified nucleic acid sequence encoding the SPIKE protein subunit S1 and S2, the RBD and RBM of SARS-COV-2 virus alone or in combination. The nucleic acid sequence preferably stem from the Wuham-Hu-1 (MN908947/NC_045512) strain or from the mutated Wuhan-Hu-1 variant, named B.1.351 or beta. Preferably, the nucleotides of this construct are DNA. Further, the nucleotides encoding the SPIKE protein subunit S1 and S2, RBD and RBM are codon optimized for optimal expression in humans.

The nucleic acid sequence located in the DNA construct may, upon administration to a subject, be expressed as a peptide or a protein in vivo in the recipient of the DNA construct. Thus, the strategy described herein takes advantage of the cellular machinery of the recipient to process the nucleotide sequence into final peptide or protein.

An advantage of the present invention is the need of treatment in the ongoing pandemic and that no other vaccines are approved for use against SARS-COV-2 virus and the subsequent disease COVID-19, which is the target of the vaccine as describe herein.

Another advantage of the described invention is the composition of the DNA vaccine with the combination of the SARS-COV-2 SPIKE sequence, codon optimization, expression in the new generation eukaryotic expression plasmid with no antibiotic resistance marker (instead the RNA-Out system is used for safety) and needle-free jet delivery to the very immunogenic skin result in protection against SARS-COV-2 infection and covid-19 disease, which is not previously seen in the art.

The target of the DNA vaccine as describe herein is the SPIKE protein, which is located on the surface of the SARS-COV-2 and is composed of the subunits S1 and S2. SPIKE enables the virus to enter the host cell of the infected subject by binding the receptor ACE2. The ACE2 receptor is directly interacting with the RBM located within the RBD area in the S1 of SPIKE.

An advantage achieved by the present invention by using SPIKE as a target for immunization is that major mutations in SPIKE are highly unlikely, as this sequence comprise enzyme cleavage sites and recognition site for ACE2, which is important for the survival of the virus. Therefore, inducing an immune response against one or more domains of SPIKE might lead to a strong protection against the virus.

Thus, a first aspect of the present invention relates to a DNA vaccine comprising a DNA construct with the nucleic acid sequence SEQ ID NO: 1 encoding a modified SPIKE protein that originates from the corona virus SARS-COV-2 or a fragment thereof having at least 80% sequence identity to SEQ ID NO: 1, preferably 90%, more preferably 95% sequence identity to SEQ ID NO: 1.

In an embodiment of the present invention the DNA vaccine comprises a fragment of the DNA construct as described herein, encoded by a nucleic acid sequence having at least 70%% sequence identity to SEQ ID NO: 1, preferably 75% such as 80% such as 85% such as 90% such as 95% such as 99% sequence identity to SEQ ID NO: 1.

Thus, another aspect of the present invention relates to a DNA vaccine comprising a DNA construct with the nucleic acid sequence SEQ ID NO: 2 encoding a modified SPIKE S1 protein that originates from the corona virus SARS-COV-2 or a fragment thereof having at least 80% sequence identity to SEQ ID NO: 2, preferably 90%, more preferably 95% sequence identity to SEQ ID NO: 2.

In an embodiment of the present invention the DNA vaccine comprises a fragment of the DNA construct as described herein, encoded by a nucleic acid sequence having at least 70% sequence identity to SEQ ID NO: 2, preferably 75% such as 80% such as 85% such as 90% such as 95% such as 99% sequence identity to SEQ ID NO: 2.

Yet, another aspect of the present invention relates to a DNA vaccine comprising a DNA construct with the nucleic acid sequence SEQ ID NO: 3 encoding a modified SPIKE S2 protein that originates from the corona virus SARS-COV-2 or a fragment thereof having at least 80% sequence identity to SEQ ID NO: 3, preferably 90%, more preferably 95% sequence identity to SEQ ID NO: 3.

In an embodiment of the present invention the DNA vaccine comprises a fragment of the DNA construct as described herein, encoded by a nucleic acid sequence having at least 70% sequence identity to SEQ ID NO: 3, preferably 75% such as 80% such as 85% such as 90% such as 95% such as 99% sequence identity to SEQ ID NO: 3.

Still, another aspect of the present invention relates to a DNA vaccine comprising a DNA construct with the nucleic acid sequence SEQ ID NO: 4 encoding a modified RBM protein that originates from the corona virus SARS-COV-2 or a fragment thereof having at least 80% sequence identity to SEQ ID NO: 4, preferably 90%, more preferably 95% sequence identity to SEQ ID NO: 4.

In one embodiment of the present invention the DNA vaccine comprises a fragment of the DNA construct as described herein, encoded by a nucleic acid sequence having at least 70% sequence identity to SEQ ID NO: 4, preferably 75% such as 80% such as 85% such as 90% such as 95% such as 99% sequence identity to SEQ ID NO: 4.

A further aspect of the present invention relates to a DNA vaccine comprising a DNA construct with the nucleic acid sequence SEQ ID NO: 5 encoding a modified RBD protein that originates from the corona virus SARS-COV-2 or a fragment thereof having at least 80% sequence identity to SEQ ID NO: 5, preferably 90%, more preferably 95% sequence identity to SEQ ID NO: 5.

In one embodiment of the present invention the DNA vaccine comprises a fragment of the DNA construct as described herein, encoded by a nucleic acid sequence having at least 70% sequence identity to SEQ ID NO: 5, preferably 75% such as 80% such as 85% such as 90% such as 95% such as 99% sequence identity to SEQ ID NO: 5.

An even further aspect of the present invention is to provide a DNA vaccine comprising a DNA construct encoding an amino acid sequence according to SEQ ID NO: 6 encoding a modified SPIKE protein that originates from the corona virus SARS-COV-2 or a fragment thereof having at least 80% sequence identity to SEQ ID NO: 6, preferably 90%, more preferably 95% sequence identity to SEQ ID NO: 6.

In one embodiment of the present invention the DNA vaccine comprises a fragment of the DNA construct as described herein, encoding an amino acid sequence having at least 70% sequence identity to SEQ ID NO: 6, preferably 75% such as 80% such as 85% such as 90% such as 95% such as 99% sequence identity to SEQ ID NO: 6.

Another aspect of the present invention is to provide a DNA vaccine comprising a DNA construct encoding an amino acid sequence according to SEQ ID NO: 7 encoding a modified SPIKE S1 protein that originates from the corona virus SARS-CoV-2 or a fragment thereof having at least 80% sequence identity to SEQ ID NO: 7, preferably 90%, more preferably 95% sequence identity to SEQ ID NO: 7.

In one embodiment of the present invention the DNA vaccine comprises a fragment of the DNA construct as described herein, encoding an amino acid sequence having at least 70% sequence identity to SEQ ID NO: 7, preferably 75% such as 80% such as 85% such as 90% such as 95% such as 99% sequence identity to SEQ ID NO: 7.

Yet another aspect of the present invention is to provide a DNA vaccine comprising a DNA construct encoding an amino acid sequence according to SEQ ID NO: 8 encoding a modified SPIKE S2 protein that originates from the corona virus SARS-COV-2 or a fragment thereof having at least 80% sequence identity to SEQ ID NO: 8, preferably 90%, more preferably 95% sequence identity to SEQ ID NO: 8.

In one embodiment of the present invention the DNA vaccine comprises a fragment of the DNA construct as described herein, encoding an amino acid sequence having at least 70% sequence identity to SEQ ID NO: 8, preferably 75% such as 80% such as 85% such as 90% such as 95% such as 99% sequence identity to SEQ ID NO: 8.

A further aspect of the present invention is to provide a DNA vaccine comprising a DNA construct encoding an amino acid sequence according to SEQ ID NO: 9 encoding a modified RBM protein that originates from the corona virus SARS-COV-2 or a fragment thereof having at least 80% sequence identity to SEQ ID NO: 9, preferably 90%, more preferably 95% sequence identity to SEQ ID NO: 9.

In one embodiment of the present invention the DNA vaccine comprises a fragment of the DNA construct as described herein, encoding an amino acid sequence having at least 70% sequence identity to SEQ ID NO: 9, preferably 75% such as 80% such as 85% such as 90% such as 95% such as 99% sequence identity to SEQ ID NO: 9.

Another aspect of the present invention is to provide a DNA vaccine comprising a DNA construct encoding an amino acid sequence according to SEQ ID NO: 10 encoding a modified RBD protein that originates from the corona virus SARS-COV-2 or a fragment thereof having at least 80% sequence identity to SEQ ID NO: 10, preferably 90%, more preferably 95% sequence identity to SEQ ID NO: 10.

In one embodiment of the present invention the DNA vaccine comprises a fragment of the DNA construct as described herein, encoding an amino acid sequence having at least 70% sequence identity to SEQ ID NO: 10, preferably 75% such as 80% such as 85% such as 90% such as 95% such as 99% sequence identity to SEQ ID NO: 10.

Another aspect of the present invention relates to a DNA vaccine comprising a DNA construct with the nucleic acid sequence SEQ ID NO: 12 encoding a modified SPIKE protein that originates from the mutated corona virus SARS-COV-2 named B.1.351 or beta or a fragment thereof having at least 80% sequence identity to SEQ ID NO: 12, preferably 90%, more preferably 95% sequence identity to SEQ ID NO: 12

In an embodiment of the present invention, the DNA vaccine comprises a fragment of the DNA construct as described herein, encoded by a nucleic acid sequence having at least 70%% sequence identity to SEQ ID NO: 12, preferably 75% such as 80% such as 85% such as 90% such as 95% such as 99% sequence identity to SEQ ID NO: 12.

Yet another aspect of the present invention relates to a DNA vaccine comprising a DNA construct with the nucleic acid sequence SEQ ID NO: 13 encoding a modified SPIKE S1 protein that originates from the mutated corona virus SARS-COV-2 named B.1.351 or beta or a fragment thereof having at least 80% sequence identity to SEQ ID NO: 13, preferably 90%, more preferably 95% sequence identity to SEQ ID NO: 13.

In an embodiment of the present invention, the DNA vaccine comprises a fragment of the DNA construct as described herein, encoded by a nucleic acid sequence having at least 70% sequence identity to SEQ ID NO: 13, preferably 75% such as 80% such as 85% such as 90% such as 95% such as 99% sequence identity to SEQ ID NO: 13.

Yet, another aspect of the present invention relates to a DNA vaccine comprising a DNA construct with the nucleic acid sequence SEQ ID NO: 14 encoding a modified SPIKE S2 protein that originates from the mutated corona virus SARS-COV-2 named B.1.351 or beta or a fragment thereof having at least 80% sequence identity to SEQ ID NO: 14, preferably 90%, more preferably 95% sequence identity to SEQ ID NO: 14.

In an embodiment of the present invention, the DNA vaccine comprises a fragment of the DNA construct as described herein, encoded by a nucleic acid sequence having at least 70% sequence identity to SEQ ID NO: 14, preferably 75% such as 80% such as 85% such as 90% such as 95% such as 99% sequence identity to SEQ ID NO: 14.

Still, another aspect of the present invention relates to a DNA vaccine comprising a DNA construct with the nucleic acid sequence SEQ ID NO: 15 encoding a modified RBM protein that originates from mutated the corona virus SARS-COV-2 name B.1.351 or beta or a fragment thereof having at least 80% sequence identity to SEQ ID NO: 15, preferably 90%, more preferably 95% sequence identity to SEQ ID NO: 15.

In one embodiment of the present invention, the DNA vaccine comprises a fragment of the DNA construct as described herein, encoded by a nucleic acid sequence having at least 70% sequence identity to SEQ ID NO: 15, preferably 75% such as 80% such as 85% such as 90% such as 95% such as 99% sequence identity to SEQ ID NO: 15.

A further aspect of the present invention relates to a DNA vaccine comprising a DNA construct with the nucleic acid sequence SEQ ID NO: 16 encoding a modified RBD protein that originates from the mutated corona virus SARS-COV-2 named B.1.351 or beta or a fragment thereof having at least 80% sequence identity to SEQ ID NO: 16, preferably 90%, more preferably 95% sequence identity to SEQ ID NO: 16.

In one embodiment of the present invention, the DNA vaccine comprises a fragment of the DNA construct as described herein, encoded by a nucleic acid sequence having at least 70% sequence identity to SEQ ID NO: 16, preferably 75% such as 80% such as 85% such as 90% such as 95% such as 99% sequence identity to SEQ ID NO: 16.

An even further aspect of the present invention is to provide a DNA vaccine comprising a DNA construct encoding an amino acid sequence according to SEQ ID NO: 17 encoding a modified SPIKE protein that originates from the mutated corona virus SARS-COV-2 named B.1.351 or beta or a fragment thereof having at least 80% sequence identity to SEQ ID NO: 17, preferably 90%, more preferably 95% sequence identity to SEQ ID NO: 17.

In one embodiment of the present invention, the DNA vaccine comprises a fragment of the DNA construct as described herein, encoding an amino acid sequence having at least 70% sequence identity to SEQ ID NO: 17, preferably 75% such as 80% such as 85% such as 90% such as 95% such as 99% sequence identity to SEQ ID NO: 17.

Another aspect of the present invention is to provide a DNA vaccine comprising a DNA construct encoding an amino acid sequence according to SEQ ID NO: 18 encoding a modified SPIKE S1 protein that originates from the mutated corona virus SARS-COV-2 named B.1.351 or beta or a fragment thereof having at least 80% sequence identity to SEQ ID NO: 18, preferably 90%, more preferably 95% sequence identity to SEQ ID NO: 18.

In one embodiment of the present invention, the DNA vaccine comprises a fragment of the DNA construct as described herein, encoding an amino acid sequence having at least 70% sequence identity to SEQ ID NO: 18, preferably 75% such as 80% such as 85% such as 90% such as 95% such as 99% sequence identity to SEQ ID NO: 18.

Yet another aspect of the present invention is to provide a DNA vaccine comprising a DNA construct encoding an amino acid sequence according to SEQ ID NO: 19 encoding a modified SPIKE S2 protein that originates from the mutated corona virus SARS-COV-2 named B.1.351 or beta or a fragment thereof having at least 80% sequence identity to SEQ ID NO: 19, preferably 90%, more preferably 95% sequence identity to SEQ ID NO: 19.

In one embodiment of the present invention, the DNA vaccine comprises a fragment of the DNA construct as described herein, encoding an amino acid sequence having at least 70% sequence identity to SEQ ID NO: 19, preferably 75% such as 80% such as 85% such as 90% such as 95% such as 99% sequence identity to SEQ ID NO: 19.

A further aspect of the present invention is to provide a DNA vaccine comprising a DNA construct encoding an amino acid sequence according to SEQ ID NO: 20 encoding a modified RBM protein that originates from the mutated corona virus SARS-COV-2 named B.1.351 or beta or a fragment thereof having at least 80% sequence identity to SEQ ID NO: 20, preferably 90%, more preferably 95% sequence identity to SEQ ID NO: 20.

In one embodiment of the present invention, the DNA vaccine comprises a fragment of the DNA construct as described herein, encoding an amino acid sequence having at least 70% sequence identity to SEQ ID NO: 20, preferably 75% such as 80% such as 85% such as 90% such as 95% such as 99% sequence identity to SEQ ID NO: 20.

Another aspect of the present invention is to provide a DNA vaccine comprising a DNA construct encoding an amino acid sequence according to SEQ ID NO: 21 encoding a modified RBD protein that originates from the mutated corona virus SARS-COV-2 named B.1.351 or beta or a fragment thereof having at least 80% sequence identity to SEQ ID NO: 21, preferably 90%, more preferably 95% sequence identity to SEQ ID NO: 21.

In one embodiment of the present invention, the DNA vaccine comprises a fragment of the DNA construct as described herein, encoding an amino acid sequence having at least 70% sequence identity to SEQ ID NO: 21, preferably 75% such as 80% such as 85% such as 90% such as 95% such as 99% sequence identity to SEQ ID NO: 21.

Another aspect of the invention relates to a DNA vaccine comprising a DNA construct with the nucleic acid sequence SEQ ID NO: 1 encoding a modified SPIKE protein that originates from the corona virus SARS-COV-2 and/or the nucleic acid sequence SEQ ID NO: 12 encoding a modified SPIKE protein that originates from SARS-COV-2 variant B.1.351 or a fragment thereof having at least 80% sequence identity to SEQ ID NO: 1 or 12, preferably 90%, more preferably 95% sequence identity to SEQ ID NO: 1 or 12.

In one embodiment of the present invention, the DNA vaccine comprises a fragment of the DNA construct as described herein, encoding an amino acid sequence having at least 70% sequence identity to SEQ ID NO: 1 or SEQ ID NO: 12, preferably 75% such as 80% such as 85% such as 90% such as 95% such as 99% sequence identity to SEQ ID NO: 1 or SEQ ID NO: 12.

Another aspect of the invention relates to a DNA vaccine comprising a DNA construct with the nucleic acid sequence SEQ ID NO: 2 encoding a modified SPIKE S1 protein that originates from the corona virus SARS-COV-2 and/or the nucleic acid sequence SEQ ID NO: 13 encoding a modified SPIKE S1 protein that originates from SARS-COV-2 variant B.1.351 or a fragment thereof having at least 80% sequence identity to SEQ ID NO: 2 or 13, preferably 90%, more preferably 95% sequence identity to SEQ ID NO: 2 or 13.

In one embodiment of the present invention, the DNA vaccine comprises a fragment of the DNA construct as described herein, encoding an amino acid sequence having at least 70% sequence identity to SEQ ID NO: 2 or SEQ ID NO: 13, preferably 75% such as 80% such as 85% such as 90% such as 95% such as 99% sequence identity to SEQ ID NO: 2 or SEQ ID NO: 13.

Another aspect of the invention relates to a DNA vaccine comprising a DNA construct with the nucleic acid sequence SEQ ID NO: 3 encoding a modified SPIKE S2 protein that originates from the corona virus SARS-COV-2 and/or the nucleic acid sequence SEQ ID NO: 14 encoding a modified SPIKE S2 protein that originates from SARS-COV-2 variant B.1.351 or a fragment thereof having at least 80% sequence identity to SEQ ID NO: 3 or 14, preferably 90%, more preferably 95% sequence identity to SEQ ID NO: 3 or 14.

In one embodiment of the present invention, the DNA vaccine comprises a fragment of the DNA construct as described herein, encoding an amino acid sequence having at least 70% sequence identity to SEQ ID NO: 3 or SEQ ID NO: 14, preferably 75% such as 80% such as 85% such as 90% such as 95% such as 99% sequence identity to SEQ ID NO: 3 or SEQ ID NO: 14.

Another aspect of the invention relates to a DNA vaccine comprising a DNA construct with the nucleic acid sequence SEQ ID NO: 4 encoding a modified RBM protein that originates from the corona virus SARS-COV-2 and/or the nucleic acid sequence SEQ ID NO: 15 encoding a modified RBM protein that originates from SARS-COV-2 variant B.1.351 or a fragment thereof having at least 80% sequence identity to SEQ ID NO: 4 or 15, preferably 90%, more preferably 95% sequence identity to SEQ ID NO: 4 or 15.

In one embodiment of the present invention the DNA vaccine comprises a fragment of the DNA construct as described herein, encoding an amino acid sequence having at least 70% sequence identity to SEQ ID NO: 4 or SEQ ID NO: 15, preferably 75% such as 80% such as 85% such as 90% such as 95% such as 99% sequence identity to SEQ ID NO: 4 or SEQ ID NO: 15.

Another aspect of the invention relates to a DNA vaccine comprising a DNA construct with the nucleic acid sequence SEQ ID NO: 5 encoding a modified RBD protein that originates from the corona virus SARS-COV-2 and/or the nucleic acid sequence SEQ ID NO: 16 encoding a modified RBD protein that originates from SARS-COV-2 variant B.1.351 or a fragment thereof having at least 80% sequence identity to SEQ ID NO: 5 or 16, preferably 90%, more preferably 95% sequence identity to SEQ ID NO: 5 or 16.

In one embodiment of the present invention, the DNA vaccine comprises a fragment of the DNA construct as described herein, encoding an amino acid sequence having at least 70% sequence identity to SEQ ID NO: 5 or SEQ ID NO: 16, preferably 75% such as 80% such as 85% such as 90% such as 95% such as 99% sequence identity to SEQ ID NO: 5 or SEQ ID NO: 16.

Another aspect of the present invention relates to a DNA vaccine comprising a DNA construct encoding an amino acid sequence according to SEQ ID NO: 6 encoding a modified SPIKE protein that originates from the corona virus SARS-CoV-2 and/or a DNA construct encoding an amino acid sequence according to SEQ ID NO: 17 encoding a modified SPIKE protein that originates from the mutated corona virus SARS-COV-2 named B.1.351 or beta or a fragment thereof having at least 80% sequence identity to SEQ ID NO: 6 or SEQ ID NO: 17, preferably 90%, more preferably 95% sequence identity to SEQ ID NO: 6 or SEQ ID NO: 17.

In one embodiment of the present invention the DNA vaccine comprises a fragment of the DNA construct as described herein, encoding an amino acid sequence having at least 70% sequence identity to SEQ ID NO: 6 or SEQ ID NO: 17, preferably 75% such as 80% such as 85% such as 90% such as 95% such as 99% sequence identity to SEQ ID NO: 6 or SEQ ID NO: 17.

Another aspect of the present invention relates to a DNA vaccine comprising a DNA construct encoding an amino acid sequence according to SEQ ID NO: 7 encoding a modified SPIKE S1 protein that originates from the corona virus SARS-CoV-2 and/or a DNA construct encoding an amino acid sequence according to SEQ ID NO: 18 encoding a modified SPIKE S1 protein that originates from the mutated corona virus SARS-COV-2 named B.1.351 or beta or a fragment thereof having at least 80% sequence identity to SEQ ID NO: 7 or SEQ ID NO: 18, preferably 90%, more preferably 95% sequence identity to SEQ ID NO: 7 or SEQ ID NO: 18.

In one embodiment of the present invention, the DNA vaccine comprises a fragment of the DNA construct as described herein, encoding an amino acid sequence having at least 70% sequence identity to SEQ ID NO: 7 or SEQ ID NO: 18, preferably 75% such as 80% such as 85% such as 90% such as 95% such as 99% sequence identity to SEQ ID NO: 7 or SEQ ID NO: 18.

Another aspect of the present invention relates to a DNA vaccine comprising a DNA construct encoding an amino acid sequence according to SEQ ID NO: 8 encoding a modified SPIKE S2 protein that originates from the corona virus SARS-CoV-2 and/or a DNA construct encoding an amino acid sequence according to SEQ ID NO: 19 encoding a modified SPIKE S2 protein that originates from the mutated corona virus SARS-COV-2 named B.1.351 or beta or a fragment thereof having at least 80% sequence identity to SEQ ID NO: 8 or SEQ ID NO: 19, preferably 90%, more preferably 95% sequence identity to SEQ ID NO: 8 or SEQ ID NO: 19.

In one embodiment of the present invention the DNA vaccine comprises a fragment of the DNA construct as described herein, encoding an amino acid sequence having at least 70% sequence identity to SEQ ID NO: 8 or SEQ ID NO: 19, preferably 75% such as 80% such as 85% such as 90% such as 95% such as 99% sequence identity to SEQ ID NO: 8 or SEQ ID NO: 19.

Another aspect of the present invention relates to a DNA vaccine comprising a DNA construct encoding an amino acid sequence according to SEQ ID NO: 9 encoding a modified RBM protein that originates from the corona virus SARS-COV-2 and/or a DNA construct encoding an amino acid sequence according to SEQ ID NO: 20 encoding a modified RBM protein that originates from the mutated corona virus SARS-COV-2 named B.1.351 or beta or a fragment thereof having at least 80% sequence identity to SEQ ID NO: 9 or SEQ ID NO: 20, preferably 90%, more preferably 95% sequence identity to SEQ ID NO: 9 or SEQ ID NO: 20.

In one embodiment of the present invention, the DNA vaccine comprises a fragment of the DNA construct as described herein, encoding an amino acid sequence having at least 70% sequence identity to SEQ ID NO: 9 or SEQ ID NO: 20, preferably 75% such as 80% such as 85% such as 90% such as 95% such as 99% sequence identity to SEQ ID NO: 9 or SEQ ID NO: 20.

Another aspect of the present invention relates to a DNA vaccine comprising a DNA construct encoding an amino acid sequence according to SEQ ID NO: 10 encoding a modified RBD protein that originates from the corona virus SARS-COV-2 and/or a DNA construct encoding an amino acid sequence according to SEQ ID NO: 21 encoding a modified RBD protein that originates from the mutated corona virus SARS-COV-2 named B.1.351 or beta or a fragment thereof having at least 80% sequence identity to SEQ ID NO: 10 or SEQ ID NO: 21, preferably 90%, more preferably 95% sequence identity to SEQ ID NO: 10 or SEQ ID NO: 21.

In one embodiment of the present invention, the DNA vaccine comprises a fragment of the DNA construct as described herein, encoding an amino acid sequence having at least 70% sequence identity to SEQ ID NO: 10 or SEQ ID NO: 21, preferably 75% such as 80% such as 85% such as 90% such as 95% such as 99% sequence identity to SEQ ID NO: 10 or SEQ ID NO: 21.

In a preferred embodiment the nucleic acid sequences according to SEQ ID NO: 1-6 stem from the Wuhan-Hu-1 (MN908947/NC_045512) strain.

In another preferred embodiment, the nuclei acid sequence according to SEQ ID NO: 12-16 stem from the mutated Wuhan-Hu-1 strain named B.1.351 or beta.

For the vaccine to induce strong immunity, both the humoral as well as the cellular immune response has to be stimulated. Humoral immunity functions against extracellular pathogenic agents and toxins. It is activated by immune cells presenting an antigen to CD4⁺ T cells on a MHC class II molecule.

Cellular immunity on the other hand, functions against intracellular pathogens. It is activated by binding of antigens to MHC Class I molecules, which is present on all nucleated cells and then presented to CD8⁺ T cells.

By stimulating both arms of the adaptive immune system, a strong immunological memory is achieved and thereby a strong protection against future infections.

Therefore, an embodiment of the present invention is to provide a DNA vaccine, wherein the proteins encoded by the sequences SEQ ID NO: 1-6 comprises an epitope that binds to MHC class I.

Another embodiment of the present invention is to provide a DNA vaccine, wherein the proteins encoded by the sequences SEQ ID NO: 1-6 comprises an epitope that binds to MHC class II.

For the immune system to be activated, the DNA construct has to be delivered into the target cells within the subject, which will then transcribe the DNA into a peptide or protein. For delivery, the DNA construct is inserted into an expression vector, which is usually a plasmid or a virus designed to control gene expression in a cell. The vector is engineered to contain regulatory sequences that act as enhancers or promotor for an efficient expression of the desired coding sequence carried by the vector. In a non-limiting example, the use of a naked circular plasmid with the key features necessary for expression, including promotor, coding sequence of interest and polyadenylation signal is provided.

Further, to enable an easy production of the plasmid, which might take place using E. coli bacteria, the plasmid comprises a selection marker. This enables production of the plasmid in a bacterium with or without using conventional bacterial resistance selection.

The eukaryotic expression vector in the DNA vaccine plasmid may contain the key elements: a minimal backbone with a strong constitutive CMV promotor, a Kozak translation initiation sequence, a polyadenylation signal, origin of replication and a selection marker for propagating the plasmid in suitable E. coli bacteria. To improve safety of the plasmid, we chose not to use antibiotic selection markers but to utilize antibiotic free RNA-OUT antisense RNA selection (an antisense RNA shutting down a suicide gene in a permissive E. coli strain; Williams 2013).

A specific and non-limiting example of a commercial available vector suitable for use in the invention as described herein is the NTC8685-eRNA41H vector provided by Nature Technology Corporation.

Thus, an embodiment of the present invention relates to an expression vector, wherein the DNA construct as described herein is inserted.

In a further embodiment, the expression vector is a eukaryotic expression vector comprising the DNA construct operationally linked to a promotor, and optionally additional regulatory sequences that regulate expression of the DNA construct.

Thus, in an embodiment of the present invention, the expression vector comprises an E. coli bacterial selection marker.

In another embodiment, the selection marker is antibiotic free RNA-OUT antisense RNA selection.

Yet in a further embodiment, the expression vector is a plasmid.

In a preferred embodiment, the expression vector comprises the following regulatory sequences; a CMV promoter, the DNA construct according to one or more of SEQ ID NO: 1-5 and/or 12-16, a Kozak translation initiation sequence, a polyadenylation signal, origin of replication and a selection marker.

In another preferred embodiment, expression vector comprises the following regulatory sequences; a CMV promoter, the DNA construct according to SEQ ID NO: 1-5 a Kozak translation initiation sequence, a polyadenylation signal, origin of replication and a selection marker.

The present invention provides a nucleotide vaccine comprising a single nucleic acid sequence encoding the SARS-Coronavirus-2 spike protein (S), preceded by a Kozak sequence and flanked by restriction enzyme-sites, enabling translation of S both in vivo and in vitro.

In a further embodiment, the Kozak translation initiation sequence has the nucleic acid sequence SEQ ID NO: 11.

In a more preferred embodiment, the expression vector is the NTC8685-eRNA41H vector.

When the vector containing the DNA construct as described herein is delivered to the target cell, the nucleotide sequence is expressed and processed to the final antigenic peptide or protein.

Thus, an embodiment of the present invention relates to the DNA vaccine as described herein for use in vaccination and/or immunization of a subject against infections and/or disease caused by SARS-COV-2.

In addition, the vaccine may comprise components normally provided together with a vaccine, and which would be known to a person skilled in the art. Such components include, but are not limited to, diluent, excipients and adjuvants.

An adjuvant comes from latin and can be translated to “help”. It is an immunological agent that improves the immune response of a vaccine. It may be added to a vaccine to boost the immune response and thereby minimize the dose of antigen needed.

Thus, in an embodiment of the present invention, the vaccine as described herein, further comprises an adjuvant.

A great variety of materials has been shown to have adjuvant activity through a variety of mechanisms. Any compound, which may increase the expression, antigenicity or immunogenicity of the present polypeptide, is a potential adjuvant.

Suitable adjuvants include but are not limited to; cytokines (e.g. GM-CSF, G-CSF, M-CSF, CSF, EPO, IL2, IL3, IL4, IL5, IL6, IL7, IL8, IL10, IL12, IL15, IL18, INFa, INFb, INFg, TGFb), growth factors, bacterial components (e.g. endotoxins including superantigens, exotoxins and cell wall components), aluminum-based salts, calcium-based salts, silica, polynucleotides, toxoids, serum proteins, vitamins, viruses, viral-derived material, poisons, venoms, imidazzoquiniline compounds, poloxamers and cationic lipids.

Administration of vaccines can be done in a number of ways as described in the following, non-limiting, examples. By intradermal injection, which is a delivery of the vaccine into the dermis of the skin, located between epidermis and the hypodermis. Alternatively, the vaccine can be administered intraveneous, which is an administration directly into the blood stream of the subject. Further, administration of the vaccine intramuscular is an injection into the muscles of the subject. In addition, the vaccine can be administered subcutaneous, which is under the skin, in the area between the muscle and the skin of the subject. Further, the vaccine can be administered intratracheal, which is administration directly into the trachea, transdermal, which is administration across the skin, Intracavity administration includes, but is not limited to administration into oral, vaginal, rectal, nasal, peritoneal, or intestinal cavities as well as, intrathecal. (i.e., into spinal canal), intraventricular (i.e., into the brain ventricles or the heart ventricles), inraatrial (i.e., into the heart atrium) and sub arachnoid (i.e., into the sub arachnoid spaces of the brain) administration.

Any mode of administration can be used as long as the mode results in the expression of the desired peptide or protein, in the desired tissue, in an amount sufficient to generate an immune response to SARS-COV-2 in a subject in need of such response.

Administration means of the present invention includes; needle injection, catheter infusion, biolistic injections, particle accelerators, needle-free jet injection, osmotic pumps, oral tablets or topical skin cream. Further, Energy assisted plasmid delivery (EAPD) methods or such methods involving the application of brief electrical pulses to injected tissues, commonly known as electroporation may be used to administer the DNA vaccine as described herein.

Optimization of the immune induction to naked DNA plasmids also involve the delivery method (Liu 2011). The inventors have found that needle-free delivery to the skin, e.g. in rabbits and pigs, improve the immune induction equally to or better than intradermal injection followed by electroporation (Borggren 2016). In agreement, others have found that needle-free delivery of DNA vaccine to the skin is superior to delivery to the muscle of e.g. pigs (Ferrari 2011). Therefore, to promote immunogenicity, a needle-free delivery of the DNA to the skin can be used. A number of needle-free delivery devices are available, which enables vaccination of both humans and animals.

In a preferred embodiment, the vaccine is administered to the subject by intradermal, intravenous, intramuscular or subcutaneous injection.

The injection of the vaccine into the subject is done using a needle-free injection method, where the skin of the subject is penetrated by a stream fluid containing the vaccine. A non-limiting example of a device fulfilling the need is the PharmaJet TROPIS® delivering system as described by the company (Document #60-10405-001 Rev.4, 2017-03-01 PharmaJet Inc., 400 Corporate Circle, Suite N, Golden, Colorado 80401 USA).

Another non-limiting example of a device fulfilling the need is the PharmaJet Stratis® jet injector delivery system (Document #60-10369-001RevA_Stratis-Product-Sheet, PharmaJet Inc., 400 Corporate Circle, Suite N, Golden, Colorado 80401 USA).

Thus, in an embodiment of the present invention, the vaccine as described herein is administered by a needle free injection.

In preferred embodiment of the present invention as described herein, the needle free injection is a needle free jet injection.

In another embodiment, the needle free injection uses a stream of fluid to penetrate the skin.

In a more preferred embodiment of the present invention, the vaccine as described herein, is administered by needle injection.

In another preferred embodiment of the present invention, the vaccine as described herein, is administered by needle injection or a needle-free injection.

The “subject” as described herein is supposed to receive the vaccine by injection and comprises humans of all ages, other primates (e.g., cynomolgus monkeys, rhesus monkeys); mammals in general, including commercially relevant mammals such as cattle, pigs, horses, sheep, goats, mink, ferrets, hamsters, cats, dogs; and/or birds. Preferred subjects are humans.

The term “subject” also includes healthy subjects of the population and, in particular, healthy subjects, who are exposed to pathogens and in need of protection against infection, such as health personnel.

Further, pathogenic infections caused by virus of the respiratory system can be particularly serious in elderly and weak patients and patients with chronic or congenital dysfunction of the respiratory system, such as asthma, cystic fibrosis, or chronic obstructive pulmonary disease (COPD).

Thus, in an embodiment of the present invention, the subject is selected from the group consisting of; humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds.

In a preferred embodiment, the subject is a human.

The vaccine as described herein may be administered in doses suitable for inducing an immune response and obtaining a sustained protective effect. In a non-limiting example, the vaccine is administered in a single dose followed by one boost, such as two boosts with two weeks apart, such as tree weeks apart.

Thus, in an embodiment of the present invention, the DNA vaccine as described herein is administered in a single dose.

In a preferred embodiment of the present invention, the DNA vaccine as described herein is administered in a single dose followed by one boost two weeks later, preferably three weeks later.

In another preferred embodiment of the present invention, the DNA vaccine as described herein is administered in a single dose followed by two boosts two weeks apart, preferably tree weeks apart.

Further, the first dose and the following boost or first and second boost as described herein does not have to be the same antigen. As seen in example 9 combining sequences from the Wuhan and the B.1.351 or beta does induce a proctetive immune response in the animal.

Thus, in one embodiment the DNA vaccine comprising anyone of the SEQ ID NO: 1-5, 12-16 or any sequences encoding the amino acid sequences according to SEQ ID NO: 6-10 or 17-21 is administered in a first dose followed by one boost two weeks later, such as two booster doses two weeks apart, wherein the booster dose comprises anyone of the SEQ ID NO: 1-5, 12-16 or any sequences encoding the amino acid sequences according to SEQ ID NO: 6-10 or 17-21.

In a second embodiment, the first dose and the following booster doses comprise the same antigens.

In another embodiment, the first dose and the following booster doses comprise different antigens.

In yet another embodiment, the first and the second booster dose comprise the same antigens.

In a further embodiment, the first and the second booster dose comprise different antigens.

In yet another embodiment, the first dose is administered as one or more doses, preferably one dose, such as two doses, such as three doses, such as four doses, such as five doses.

In a further embodiment, the booster dose is administered as one or more doses, preferably one dose, such as two doses, such as three doses, such as four doses, such as five doses.

In yet a further embodiment, the first dose is administered as one or more doses comprising one or more DNA constructs with anyone of the nucleic acid sequences SEQ ID NO: 1-5 or any sequences encoding the amino acid sequences according to SEQ ID NO: 6-10 and/or one or more DNA constructs with anyone of the nucleic acid sequences SEQ ID NO: 12-16 or any sequences encoding the amino acid sequences according to SEQ ID NO: 17-21.

In another embodiment, the booster doses are administered as one or more doses comprising the same or different DNA constucts with anyone of the nucleic acid sequences SEQ ID NO: 1-5 or any sequences encoding the amino acid sequences according to SEQ ID NO: 6-10 and/or with anyone of the nucleic acid sequences SEQ ID NO: 12-16 or any sequences encoding the amino acid sequences according to SEQ ID NO: 17-21.

Further, the size of each dose of the plasmid DNA vaccine, including optional booster doses, has to be suitable for inducing an immune response and obtaining a sustained protective effect. Non-limiting examples of doses is 1 mg, such as 2 mg, such as 3 mg, such as 4 mg, such as 5 mg.

Thus, in an embodiment of the present invention the DNA vaccine is administered in a dose of 0.5-5 mg, such as 1 mg, preferably in a dose of 2 mg, more preferably in a dose of 3 mg, more preferably 4 mg, even more preferably 5 mg.

In another an embodiment of the present invention, the DNA vaccine is administered in a dose of 0.5-5 mg, such as at least 0.5, such as at least 1 mg, preferably in a dose of at least 2 mg, more preferably in a dose of at least 3 mg, more preferably in a dose of at least 4 mg, even more preferably in a dose of at least 5 mg.

In a further embodiment, the DNA vaccine is administered in a dose in the range of 0.5-5 mg, such as in the range of 1-5 mg, such as in the range of 2-5 mg, such as in the range of 3-5 mg, such as in the range of 4-5 mg.

The invention further relates to a pharmaceutical composition for use as a medicament.

Thus, in on aspect the invention relates to a pharmaceutical composition comprising the DNA construct inserted into the vector according to anyone of the preceding aspects or embodiments.

In one embodiment, the composition according to the invention is effective against any genotypic variant of SARS-COV-2.

In another embodiment, DNA vaccine according to the invention, for use in the preparation of a medicament for inducing a protective immune response to SARS-CoV-2.

Another aspect of the invention relates to a method for inducing a protective immune response to SARS-COV-2 comprising; administering said composition according to the invention to a subject by intradermal, intravenous, intramuscular or subcutaneous injection or by inhalation.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1: Construction of the DNA Vaccine

The SPIKE-encoding DNA vaccine sequence was synthetically synthezised as codon optimized, double-stranded DNA sequence, based on unmodified, wildtype full-length SARS-COV-2 SPIKE protein sequence from strain Wuhan-Hu-1 (MN908947/NC_045512). The SPIKE-encoding DNA vaccine sequence is preceded by a Kozak sequence and followed by stop codon, this entity is flanked by unique restriction enzyme sites to facilitate transfer to other vectors, such as but not limited to, NTC8685-eRNA41H.

This sequence was cloned into NTC8685-eRNA41H (Nature Technologies Corporation, Lincoln, NE, USA). NTC8685-eRNA41H is a nano-plasmid eukaryotic expression vector that uses an antisense RNA sucrose selection method (RNA-OUT™) instead of antibiotic-resistance selection. Nature Technologies produced the DNA vaccine. The construct was sequenced and tested for expression prior to use. The DNA vaccine, supplied as 10 mg/ml or 5 mg/ml in PBS, was prepared as a 5 mg/ml in sterile PBS (without MgCl₂/CaCl₂)).

### Example 2: Vaccination in Animal Models

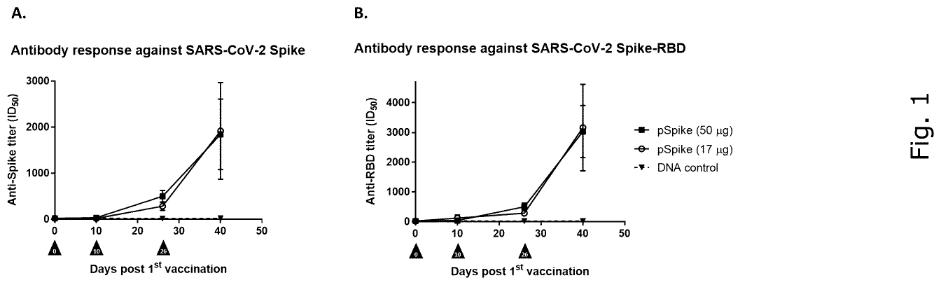

The immune response against the DNA vaccine is examined in an animal model (mice). 15 mice are divided into three groups, with 5 mice in each (CB6F1 mice, 7 weeks old females). On day 0, 10 and 26, group I and II receive 50 μg or 17 μg, respectively of the SPIKE S1+S2 vaccine in PBS/water, while the third group receives 50 μg of non-coronavirus DNA vector vaccine. On day 0, 10, 26 and 40 serum is obtained from each mouse and antibody titers are determined by ELISA. FIG. 1A: Antibody titers obtained against SARS-COV-2 SPIKE S1+S2 protein. FIG. 1B: Antibody titers obtained against the receptor-binding domain (RBD) of SARS-CoV-2 SPIKE protein.

The nucleotide sequence encoding the SARS-COV-2 SPIKE protein gives rise to an immunogenic protein product. In mice, we show that this sequence generates a strong, specific antibody response against the SARS-COV-2 SPIKE protein as well as the SPIKE receptor-binding domain (RBD) (FIG. 1A & B).

### Example 3: Neutralizing Antibodies

The immune response against the DNA vaccine is examined in an animal model (mice). 15 mice are divided into three groups, with 5 mice in each (CB6F1 mice, 7 weeks old females). On day 0, 10 and 26, group I and group II received 50 μg or 17 μg of the SPIKE S1+S2 vaccine in PBS/water respectively, while the third group received 50 μg of non-coronavirus DNA vector vaccine. On day 40, serum was obtained and neutralization titers were determined in a microneutralization test using a Danish SARS-COV-2 isolate. Human SARS-COV-2 convalescence plasma were used as a titer reference to determine functional range of neutralization.

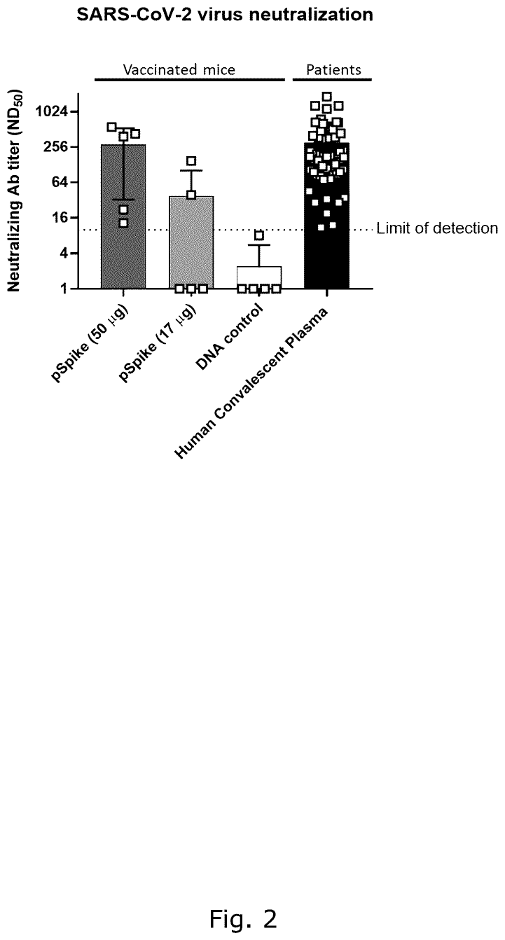

The nucleotide sequence encoding the SARS-COV-2 SPIKE protein gives rise to neutralizing antibodies. Antibodies elicited against spike, especially the RBD region, are expected to hinder the virus to bind to its receptor (ACE-2), thereby prevent infection. Indeed, serum from mice immunized with the naked DNA vaccine, neutralizes SARS-COV-2 wildtype virus at titers equivalent to human convalescence sera (FIG. 2).

### Example 4: Cellular Immune Response

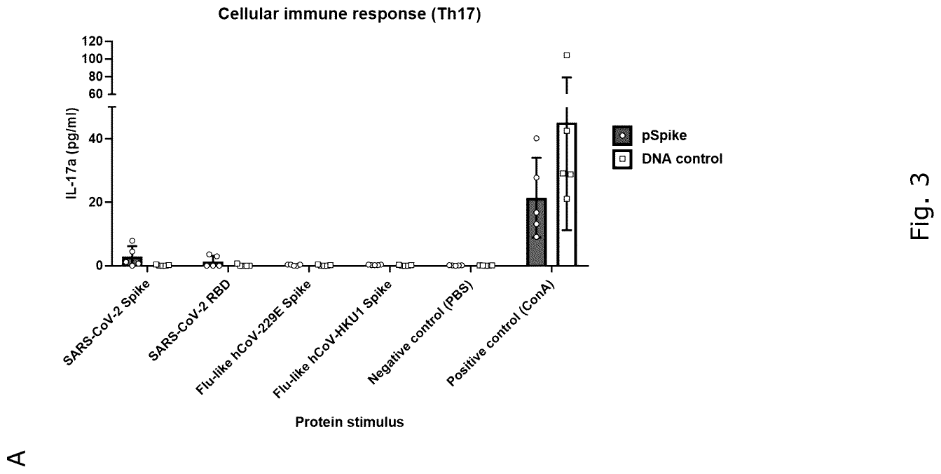

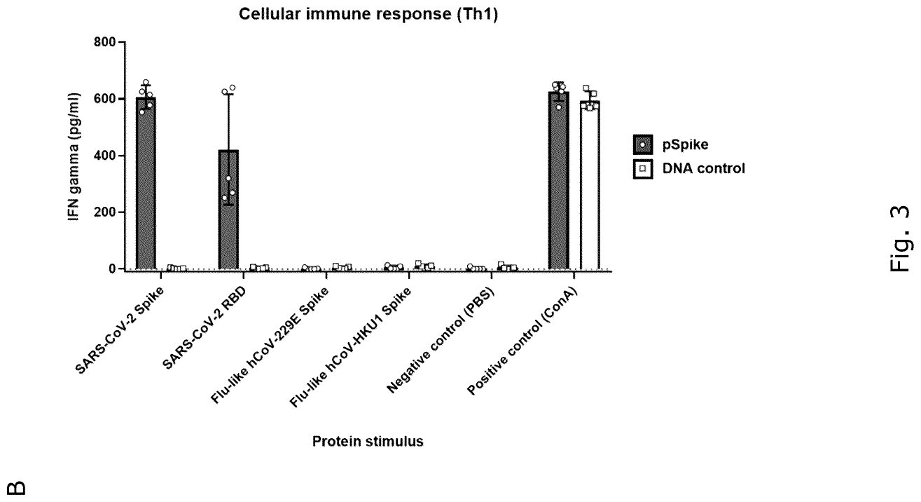

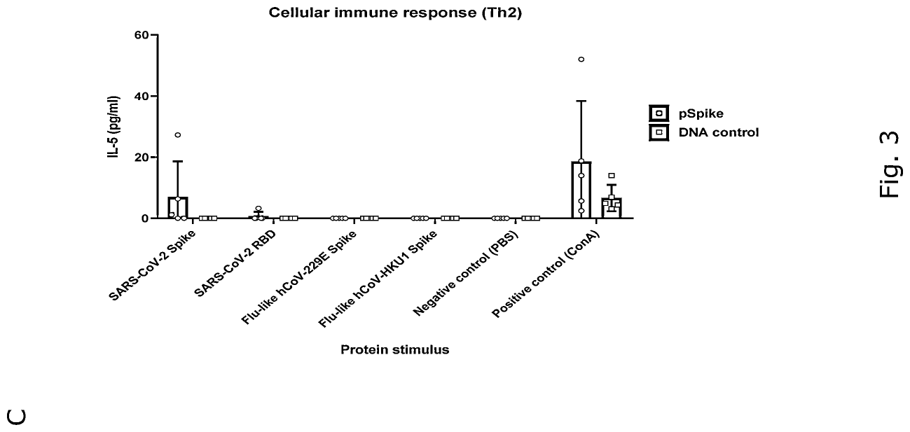

The cellular immune response induced by SARS-COV-2 DNA vaccine in mice was examined. 10 mice were divided into two groups, with 5 mice in each ((CB6F1 mice, 7 weeks old females)). On day 0, 10 and 26, the group I received 50 μg of the SPIKE S1+S2 vaccine in PBS/water, while the second group received 50 μg of non-coronavirus DNA vector vaccine. On day 40 the spleen was harvested from each mouse. Cellular immuneresponse was measured by restimulation of the spleenocytes with, SARS-COV-2 SPIKE. SARS-COV-2 RBD, hCoV-HKU1 SPIKE, hCoV-229E SPIKE, PBS (negative control) or Concanavalin A (positive control), respectively, followed by measuring of the cytokine production by cytokine-specific ELISA. FIG. 3A: INF-gamma production corresponding to a Th1 response. FIG. 3B: IL-5 production corresponding to a Th2 response. FIG. 3C: IL17a production corresponding to a Th17 response.

DNA vaccines are known to bias the activation of T-helper cell response, favoring the activation of the Th1 phenotype. We found that the naked DNA vaccine encoding the SARS-COV-2 SPIKE also shows a preferred Th1-response. Mice were immunized three times with the DNA vaccine and spleens were isolated 2 weeks after the last immunization. The spleenocytes were then re-stimulated with SARS-CoV-2 SPIKE protein or SPIKE-RBD, to trigger a Th-cell response, and Th-phenotype-specific cytokines were measured. As expected, a dominating Th1-response was detected, with lower Th2 and Th17 responses (FIG. 3A-C).

In addition, re-stimulation with other commonly circulating human (common cold-like) coronaviruses, such as hCoV-229E and hCoV-HKU1, did not re-activate the spleenocytes, indicating a specific SARS-COV-2 response to the DNA vaccine (FIG. 3A-C).

Examples with Rhesus Macaques

### Animals and Study Design

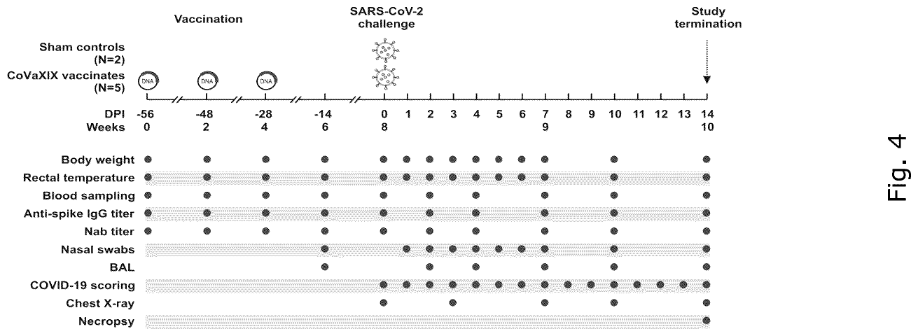

Seven male and female adult rhesus macaques (Macaca mulatta), 2 to 8 years old (mean: 4 years), were randomly divided into two groups: CoVaXIX vaccinates (N=5) and sham controls (N=2). Animals received three immunizations of 2 mg DNA each at weeks 0, 2, and 4 (FIG. 4). The unadjuvanted vaccine was administered via the intradermal route with four 100 μL doses per immunization, equally distributed over the left and right scapul4 region. At week 8, all eight animals were challenged with 1.0×10⁵ TCID₅₀ (1.2×10⁸ RNA copies, 1.1×10⁴ PFU) SARS-COV-2 (strain nCOV-WAI-2020; MN985325.1). The virus was administered as 1 mL by the intranasal (IN) route and 1 mL by the intra-tracheal (IT) route. The animals were housed at Bioqual Inc. (Rockville, MD). All animal studies were conducted in compliance with relevant local, state, and federal regulations and were approved by the Institutional Animal Care and Use Committee (IACUC).

### Enzyme-Linked Immunosorbent Assay (ELISA)

SARS-COV-2 SPIKE protein-specific IgG in serum was quantified by enzyme-linked immunosorbent assay (ELISA). In brief, microtiter plates were coated with 1 μg/mL SARS-COV-2 SPIKE protein (Sino Biological Inc., USA) in 1× PBS and incubated overnight at 4° C. Plates were washed once with wash buffer (0.05% Tween20 in 1× DPBS) and blocked with 350 μL Casein in PBS for 2 hours at room temperature. The block solution was discarded and serial dilutions of serum in casein in PBS added to the wells, followed by a 1 hour incubation at room temperature. Plates were washed three times with wash buffer and incubated for 1 hour at room temperature with a 1:1000 dilution of anti-macaque IgG HRP (NIH NHP Reagent Program). Plates were washed three times with wash buffer followed by addition of 100 μL of SeraCare KPL TMB SureBlue Start solution. The reaction was stopped after 5-10 minutes with the addition of 100 μL SeraCare KPL TMB Stop solution per well. The absorbance was measured at 450 nm using 620 nm as a reference. ELISA endpoint titers were defined as the highest reciprocal serum dilution that yielded an absorbance >0.2. Log₁₀ endpoint titers are reported.

### Plaque Reduction Neutralization Test (PRNT)

The PRNT was performed in 6-well tissue culture plates seeded with 1.75×10⁵ Vero76 cells/well the day before. Serum samples were heat-inactivated at 56° C. for 30 minutes and tested in duplicate in a three-fold serial dilution ranging from 1:20 to 1:4860. Each serum dilution was pre-incubated with 30 PFU SARS-COV-2 (challenge strain) for 1 hour at 37° C. before addition to the Vero76 monolayers. After an incubation of 1 hour at 37° C., the supernatants containing the serum/virus mixture were removed and the monolayer washed once with PBS before overlaying with a semi-solid culture medium. Following a three-day incubation at 37° C. 5% CO₂, the cells were fixed and stained with crystal violet as described. The reciprocal of the serum dilutions causing plaque reductions of 90% (PRNT90) and 50% (PRNT50) were recorded as titers.

### Subgenomic SARS-COV-2 RNA Assay

Replicating SARS-COV-2 virus was detected and measured using a real-time RT-PCR assay targeting viral replication cellular intermediates not packaged into virions. In particular, the SARS-COV-2 E gene subgenomic messenger RNA (sgmRNA) was targeted using a leader-specific primer with primers and probes targeting sequences downstream of the start codons of the E gene.

### Statistical Analyses

Variation in paired continuous variables were compared between time points using the non-parametric Friedman test with Dunn's correction for multiple comparisons. All statistical analyses and graphing were done with GraphPad PRISM version 8.0.2. (GraphPad Software Inc., San Diego, CA).

### Example 5—Immunogenicity in Rhesus Macaques

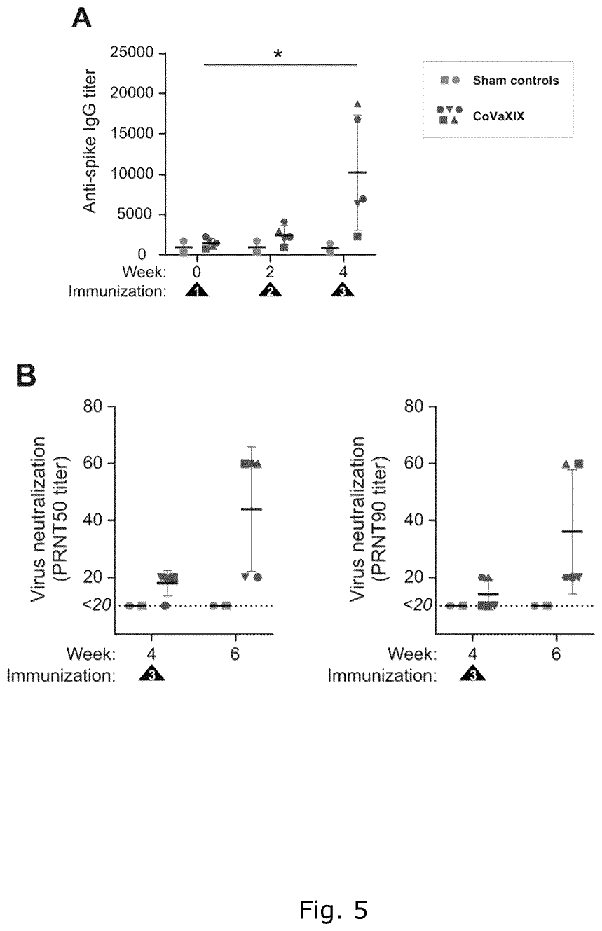

The immune response induced by SARS-COV-2 DNA vaccine in non-human primates was tested. Seven Rhesus macaques (2 to 8 years old) were divided into two groups, wherein five received the SARS-COV-2 DNA vaccine and 2 received a sham control. The animals received three immunizations of 2 mg DNA without adjuvant by intradermal route at week 0, 2 and 4 (FIG. 4; see also Animals and study design). SPIKE-specific binding antibodies were observed by ELISA in Rhesus macaques vaccinated with the SARS-COV-2 DNA vaccine after the second immunization (Week 4, FIG. 5A), and levels were significantly increased after a third vaccination.

### Example 6—Neutralizing Antibody Response

Neutralizing antibody (Nab) responses in the seven rhesus macaques vaccinated according to example 5 and FIG. 4 were evaluated using a live virus plaque reduction neutralization test (PRNT). The PRNT was performed in 6-well tissue culture plates seeded with 1.75×10⁵ Vero76 cells/well the day before (see also Animals and study design). Serum samples were heat-inactivated at 56° C. for 30 minutes and tested in duplicate in a three-fold serial dilution ranging from 1:20 to 1:4860. Each serum dilution was pre-incubated with 30 plaque forming units (PFU) SARS-COV-2 (challenge strain) for 1 hour at 37° C. before addition to the Vero76 monolayers. After an incubation of 1 hour at 37° C., the supernatants containing the serum/virus mixture were removed and the monolayer washed once with PBS before overlaying with a semi-solid culture medium. Following a three-day incubation at 37° C. 5% CO₂, the cells were fixed and stained with crystal violet. The reciprocal of the serum dilutions causing plaque reductions of 90% (PRNT90) and 50% (PRNT50) were recorded as titers.

Nabs capable of reducing PFU by more than 50% in the PRNT at a serum dilution greater than 1:20 (PRNT50=20, median 20) were observed in four of five Rhesus macaques vaccinated with the SARS-COV-2 DNA vaccine after the second immunization (FIG. 5B). Nab responses were boosted by the third immunization, with all Rhesus macaques vaccinated with the SARS-COV-2 DNA vaccine having developed Nabs by week 6, measured as PRTN50 (median PRNT50=60) and the more stringent PRNT90 (median:20) two weeks before virus challenge (FIG. 5B).

### Example 7—Protective Efficacy of the SARS-COV-2 DNA Vaccine

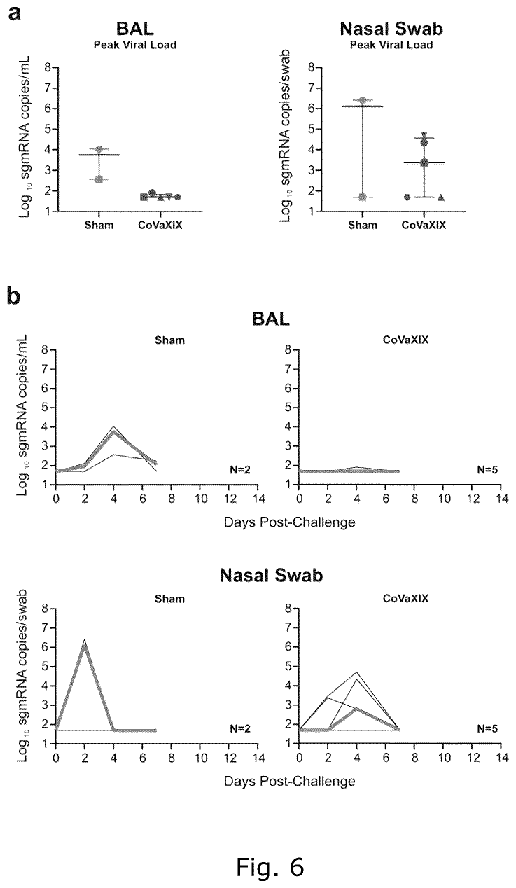

The protective efficacy of the SARS-COV-2 DNA vaccine was evaluated. The seven rhesus macaques were vaccinated according to example 5 and FIG. 4. At week 8, four weeks after final immunization, all animal were challenged with 1.0×10⁵ TCID50 SARS-COV-2 by intranasal and intratracheal routes. SARS-COV-2 virus was measured in bronchoalveolar lavage (BAL) and nasal swabs using an RT-PCR specific for subgenomic mRNA (sgmRNA), which are cellular intermediates and believed to represent replicating virus. Both sham controls were infected and showed a median peak of 3.74 log₁₀ sgmRNA copies/mL in BAL (FIG. 6A). The vaccinated animals had a 2.04 log₁₀ reduction in viral RNA in BAL. In particular, four out of five animals had viral loads below the quantitation limit of the assay (1.69 log₁₀ sgmRNA copies/mL), one animal had a detectable low peak of 1.91 log 10 sgmRNA copies/mL on day 4 post-challenge (FIG. 6A).

The median peak viral load in the nasal swabs was 3.37 log₁₀ sgmRNA copies/mL, representing a 2.73 log₁₀ reduction in viral RNA relative to the median viral load of the sham controls (6.10 log₁₀ sgmRNA copies/mL) (FIG. 6B). Since only one of the two sham controls had detectable viral load in the nasal swab, the vaccinated group was further compared to sham controls (N=10 and N=20) from two independent studies performed at the same facility with the same input virus (Mercardo et al, Guebre-Xabier et al). In these studies, animals had median peak viral loads of 6.82 and 5.59 log₁₀ sgmRNA in nasal swabs. Compared to these data, Rhesus macaques vaccinated with the SARS-COV-2 DNA vaccine had a 3.34 and 2.22 log₁₀ reduction in viral RNA.

### Example 8—Immunogenicity Profiles of Two Different SARS-COV-2 SPIKE DNA Vaccines

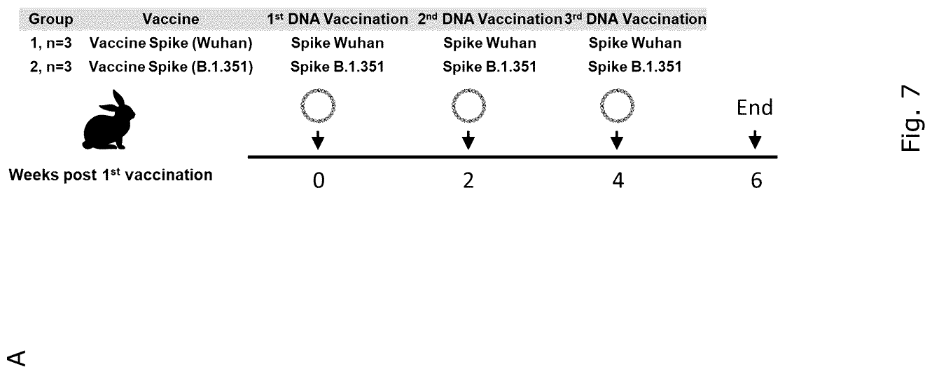

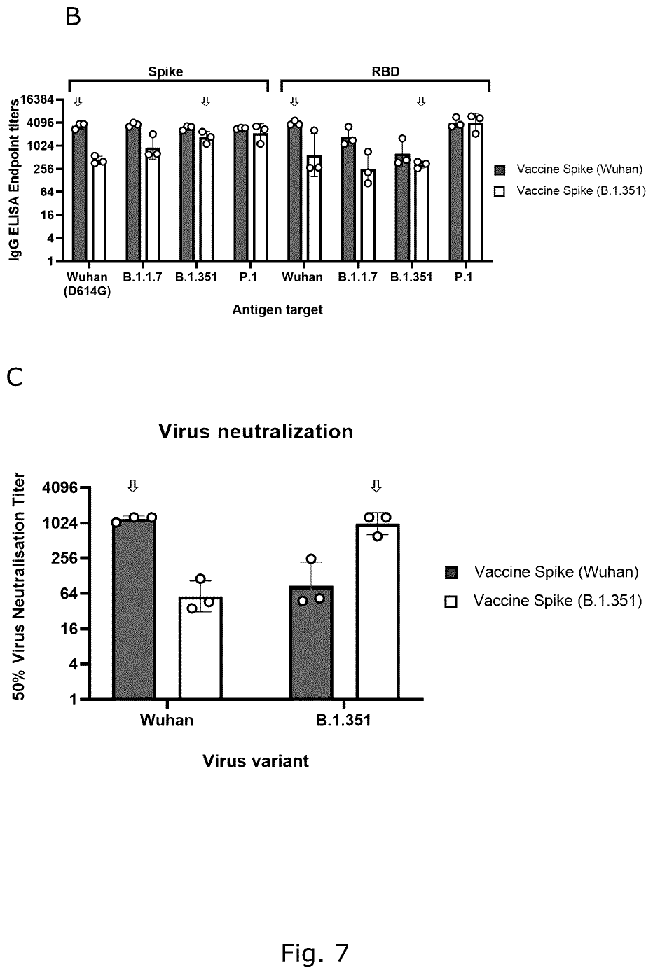

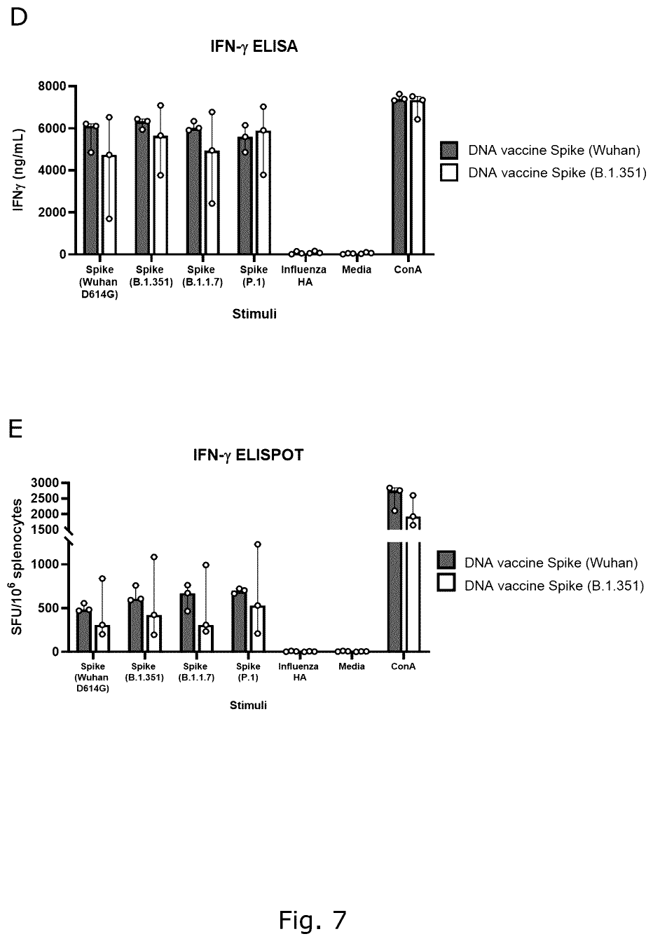

The immunogenicity of DNA vaccines based on either the original (Wuhan) SPIKE sequence (SEQ ID NO 1) or the Spike B.1.351 sequence (SEQ ID NO 12) were evaluated. The SPIKE B. 1.351 differs in 11 amino acid positions (deletions or substitutions) compared to the SPIKE Wuhan, including 3 key substitutions in the RBD (K417N, E484K and N501Y). Two groups of three New Zeeland White rabbits were immunized with 125 μg of SPIKE-Wuhan or SPIKE-B. 1.351 DNA vaccine, respectively. Each group received three immunizations, two weeks apart (regiment; FIG. 7A).

Two weeks after the third immunization, sera were serially diluted and assessed by IgG ELISA for antibody levels against SPIKE or RBD proteins from four different SARS-COV-2 variants: Wuhan, B.1.351, B.1.1.7 and P.1. The Wuhan SPIKE protein used in the ELISA contains mutation D614G, which is one of the first mutations fixed in the original virus and is now present in the majority of all circulating sequences. This mutation is located outside of the RBD.

Both vaccines elicited an antibody response against all SPIKE and RBD variants (FIG. 7B). The SPIKE-Wuhan vaccine showed robust levels of antibody response against all four SPIKE variants tested, while the SPIKE B. 1.351 vaccine showed a slight bias towards the B. 1.351 and the more similar P.1 SPIKE proteins. Titers are shown as the geometric mean with a geometic standared deviation. White arrows indicate homologous antibody-antigen response.

The virus neutralization capacity of antibodies raised against the two vaccines were evaluated in an in-house, live virus microneutralization assay. A Wuhan-like, Danish isolate from early 2020 and a B.1.351 Danish isolate from 2021 were used.

Despite similar high levels of anti-SPIKE B.1.351 elicited by the two vaccines (FIG. 7B), only antibodies raised against the SPIKE-B.1.351 efficiently neutralized the B.1.351 virus (FIG. 7C). Similarily, antibodies raised against the SPIKE-Wuhan vaccine efficiently neutralized the Wuhan virus, while sera from SPIKE-B.1.351 vaccinated animals could not neutralize the virus to the same level (FIG. 7C).

The cell mediated immune response was measured by IFN-γ ELISA (FIG. 7D) and IFN-γ ELISPOT (FIG. 7E) assays. Spenocytes from vaccinated animals were isolated two weeks after the third immunization and re-stimulated with homologous and heterologous SPIKE proteins. If the cells recognize the stimuli, they get re-activated and produce interferon gamma (IFN-γ), which is then used as a read out in both assays. Both vaccines induce a broad, SPIKE-specific, cell mediated immune reponse (FIG. 7D-E). An unrelated influenza HA protein was used as a (negative) control for unspecific immune response. Cell media was used as a negative stimuli control and Concanavalin A as a positive stimuli control.

### Example 9—Heterologous Boost

It should be possible to broaden the antibody repertoire against a specific pathogen by priming with one vaccine and boosting with another. The SPIKE-B.1.351 protein contains at least three known mutations/epitopes, which are associated with reduced neutralization by antibodies raised against SARS-COV-2 Wuhan infection or vaccination. A boost with a SPIKE-B.1.351 vaccine could therefore be beneficial to broaden an existing wildtype SARS-COV-2 response.

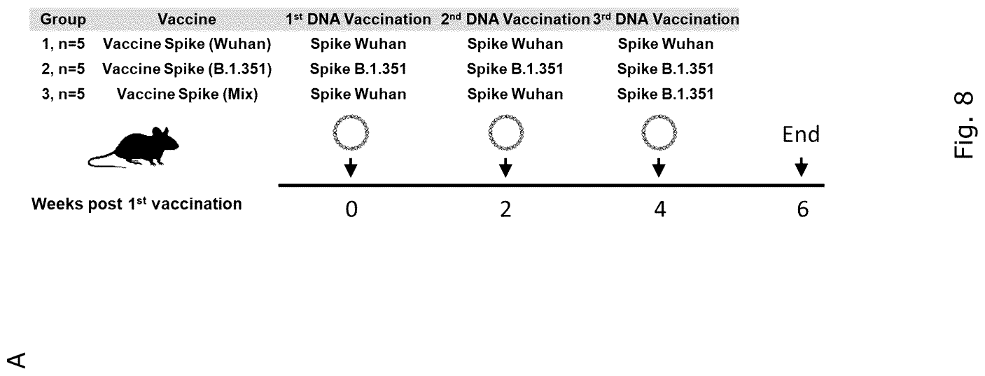

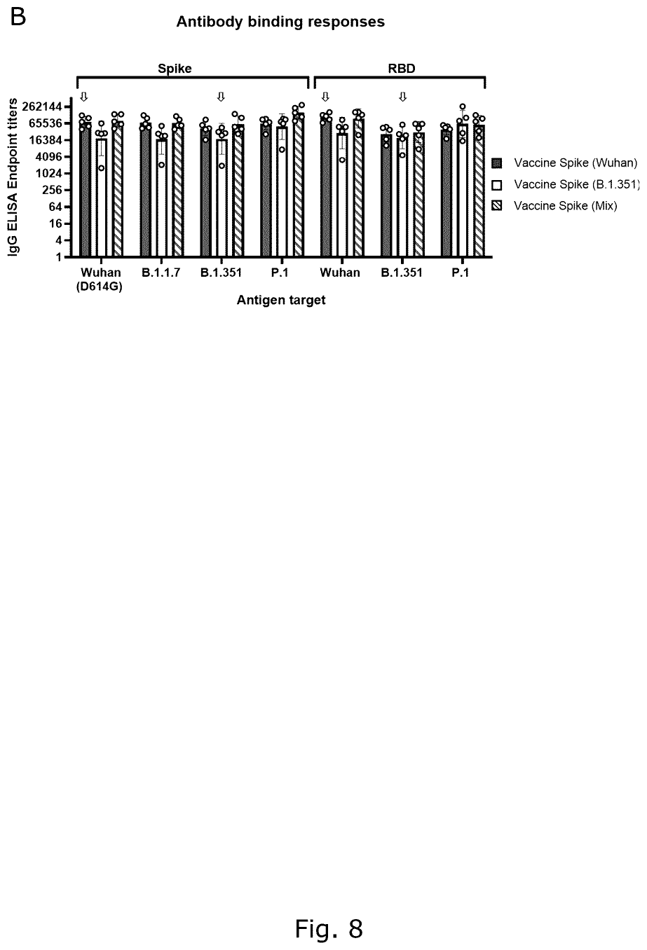

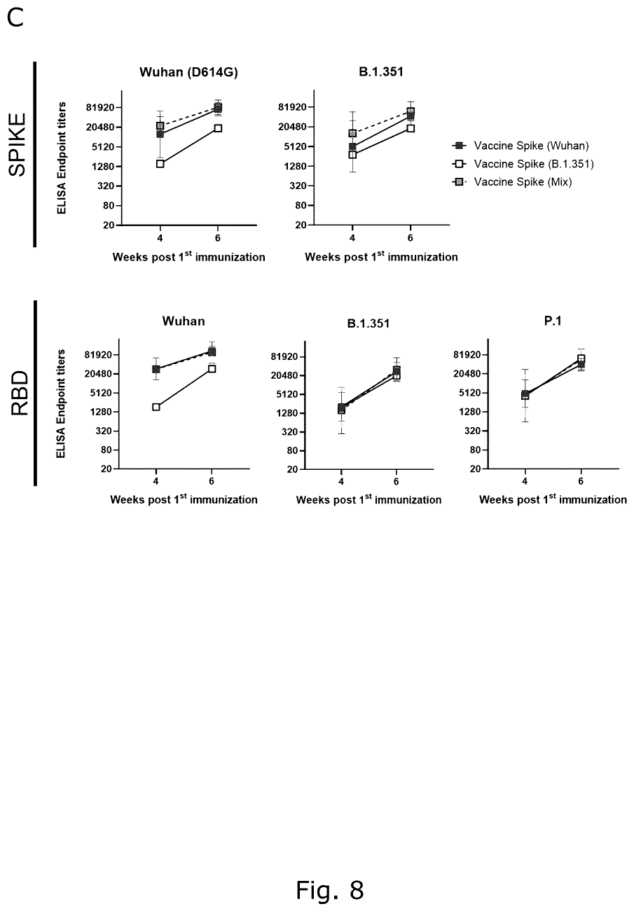

The effect of a heterologous DNA vaccine boost was assessed using three groups of mice. The first group received three immunizations with the SPIKE-Wuhan vaccine, the second group received three immunizations with the SPIKE-B.1.351 vaccine and the third group received two immunizations with the SPIKE-Wuhan vaccine followed by a boost with the SPIKE-B.1.351 vaccine (FIG. 8A). Mouse sera were isolated two weeks after the third immunization and analyzed with IgG ELISA. To evaluate the broadness of the response, four SPIKE variants and three RBD variants were used. The heterologous boost induced the same antibody response, or higher, against all SPIKE-antigens tested, compared to the response from the homologous immunizations (FIG. 8B). White arrows indicate homologous antibody-antigen response

To further explore the dynamics of the boosts, IgG titers obtained at week 4 and 6 were compared (FIG. 8C). These data confirm that a boost is beneficial in terms of antibody titers and that a heterologous boost give rise to the same or higher levels of antibodies than the homologous boost.

### REFERENCES

Borggren M, Nielsen, J, Karlsson I, et al. A polyvalent influenza DNA vaccine applied by needle-free intradermal delivery induces cross-reactive humoral and cellular immune responses in pigs. Vaccine 2016; 34:3634-3540.
Ferrari L, Borghetti P, Gozio S, et al. Evaluation of the immune response induced by intradermal vaccination by using a needle-less system in comparison with the intramuscular route in conventional pigs. Res Vet Sci. 2011; 90:64-71.
Jones S, Evans K, McElwaine-Johnn H, et al. DNA vaccination protects against an influenza challenge in a double-blind randomized placebo-controlled phase 1b clinical trial. Vaccine 2009; 27:2506-2512.
Kutzler M & Weiner D. DNA vaccines: Ready for prime time? Nature Review 2008; 9:776-788.
Liu MA. DNA vaccines: An historical perspective and view to the future. Immunol Rev 2011; 239:62-84.
Rosa DS, de Souza A J, Boscardin SB. DNA vaccines: how much have we accomplished in the last 25 years? J Vaccines Vaccin, 2015; 6:283.
Williams J. Vector design for improved DNA vaccine efficacy, safety and production. Vaccines 2013; 1:225-249.
Mercado, N. B. et al., Single-shot Ad26 vaccine protects against SARS-COV-2 in rhesus macaques. Nature (2020). doi: 10.1038/s41586-020-2607-z.
Guebre-Xabier, M. et al. Title: NVX-CoV2373 vaccine protects cynomolgus macaque upper and lower airways 3 against SARS-COV-2 challenge 4 5 Authors and Affiliations: 6. bioRxiv 2020.08.18.256578 (2020). doi: 10.1101/2020.08.18.256578.

Sequence listing

SEQ ID NO: 1 (nucleic acid sequence for SARS-COV-2 SPIKE protein,
codon optimized for humans)
ATGTTCGTGTTTCTGGTGCTGCTGCCTCTGGTGTCCAGCCAGTGTGTGAACCTGACCACC
AGAACACAGCTGCCTCCAGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCCACGTGTCCGGCACCAATGGCACCAAGAGATTCGA
CAACCCCGTGCTGCCCTTCAACGACGGGGTGTACTTTGCCAGCACCGAGAAGTCCAACA
TCATCAGAGGCTGGATCTTCGGCACCACACTGGACAGCAAGACCCAGAGCCTGCTGATC
GTGAACAACGCCACCAACGTGGTCATCAAAGTGTGCGAGTTCCAGTTCTGCAACGACCC
CTTCCTGGGCGTCTACTACCACAAGAACAACAAGAGCTGGATGGAAAGCGAGTTCCGGG
TGTACAGCAGCGCCAACAACTGCACCTTCGAGTACGTGTCCCAGCCTTTCCTGATGGACC
TGGAAGGCAAGCAGGGCAACTTCAAGAACCTGCGCGAGTTCGTGTTCAAGAACATCGAC
GGCTACTTCAAGATCTACAGCAAGCACACCCCTATCAACCTCGTGCGGGATCTGCCTCAG
GGCTTCTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGCGGATG
GACAGCTGGTGCCGCCGCTTACTATGTGGGCTACCTGCAGCCTAGAACCTTCCTGCTGA
AGTACAACGAGAACGGCACCATCACCGACGCCGTGGATTGTGCTCTGGATCCTCTGAGC
GAGACAAAGTGCACCCTGAAGTCCTTCACCGTGGAAAAGGGCATCTACCAGACCAGCAA
CTTCCGGGTGCAGCCCACCGAATCCATCGTGCGGTTCCCCAATATCACCAATCTGTGCCC
CTTCGGCGAGGTGTTCAATGCCACCAGATTCGCCTCTGTGTACGCCTGGAACCGGAAGC
GGATCAGCAATTGCGTGGCCGACTACTCCGTGCTGTACAACTCCGCCAGCTTCAGCACCT
TCAAGTGCTACGGCGTGTCCCCTACCAAGCTGAACGACCTGTGCTTCACAAACGTGTACG
CCGACAGCTTCGTGATCCGGGGAGATGAAGTGCGGCAGATTGCCCCTGGACAGACAGG
CAAGATCGCCGACTACAACTACAAGCTGCCCGACGACTTCACCGGCTGTGTGATTGCCT
GGAACAGCAACAACCTGGACTCCAAAGTCGGCGGCAACTACAATTACCTGTACCGGCTG
TTCCGGAAGTCCAATCTGAAGCCCTTCGAGCGGGACATCTCCACCGAGATCTATCAGGC
CGGCAGCACCCCTTGTAACGGCGTGGAAGGCTTCAACTGCTACTTCCCACTGCAGTCCTA
CGGCTTTCAGCCCACAAATGGCGTGGGCTATCAGCCCTACAGAGTGGTGGTGCTGAGCT
TCGAACTGCTGCATGCCCCTGCCACAGTGTGCGGCCCTAAGAAAAGCACCAATCTCGTG
AAGAACAAATGCGTGAACTTCAACTTCAACGGCCTGACCGGCACCGGCGTGCTGACAGA
GAGCAACAAGAAGTTCCTGCCATTCCAGCAGTTTGGCCGGGATATCGCCGATACCACAG
ACGCCGTTAGAGATCCCCAGACACTGGAAATCCTGGACATCACCCCTTGCAGCTTCGGC
GGAGTGTCTGTGATCACCCCTGGCACCAACACCAGCAATCAGGTGGCAGTGCTGTACCA
GGACGTGAACTGTACCGAAGTGCCCGTGGCCATTCACGCCGATCAGCTGACACCTACAT
GGCGGGTGTACTCCACCGGCAGCAATGTGTTTCAGACCAGAGCCGGCTGTCTGATCGGA
GCCGAGCACGTGAACAATAGCTACGAGTGCGACATCCCCATCGGCGCTGGCATCTGTGC
CAGCTACCAGACACAGACAAACAGCCCCAGACGGGCCAGATCTGTGGCCAGCCAGAGCA
TCATTGCCTACACAATGTCTCTGGGCGCCGAGAACAGCGTGGCCTACTCCAACAACTCTA
TCGCTATCCCCACCAACTTCACCATCAGCGTGACCACAGAGATCCTGCCTGTGTCCATGA
CCAAGACCAGCGTGGACTGCACCATGTACATCTGCGGCGATTCCACCGAGTGCTCCAAC
CTGCTGCTGCAGTACGGCAGCTTCTGCACCCAGCTGAATAGAGCCCTGACAGGGATCGC
CGTGGAACAGGACAAGAACACCCAAGAGGTGTTCGCCCAAGTGAAGCAGATCTACAAGA
CCCCTCCTATCAAGGACTTCGGCGGCTTCAATTTCAGCCAGATTCTGCCCGATCCTAGCA
AGCCCAGCAAGCGGAGCTTCATCGAGGACCTGCTGTTCAACAAAGTGACACTGGCCGAC
GCCGGCTTCATCAAGCAGTATGGCGATTGTCTGGGCGACATTGCCGCCAGGGATCTGAT
TTGCGCCCAGAAGTTTAACGGACTGACAGTGCTGCCTCCTCTGCTGACCGATGAGATGAT
CGCCCAGTACACATCTGCCCTGCTGGCCGGCACAATCACAAGCGGCTGGACATTTGGAG
CTGGCGCCGCTCTGCAGATCCCCTTTGCTATGCAGATGGCCTACCGGTTCAACGGCATC
GGAGTGACCCAGAATGTGCTGTACGAGAACCAGAAGCTGATCGCCAACCAGTTCAACAG
CGCCATCGGCAAGATCCAGGACAGCCTGAGCAGCACAGCAAGCGCCCTGGGAAAGCTG
CAGGACGTGGTCAACCAGAATGCCCAGGCACTGAACACCCTGGTCAAGCAGCTGTCCTC
CAACTTCGGCGCCATCAGCTCTGTGCTGAACGATATCCTGAGCAGACTGGACAAGGTGG
AAGCCGAGGTGCAGATCGACAGACTGATCACCGGAAGGCTGCAGTCCCTGCAGACCTAC
GTTACCCAGCAGCTGATCAGAGCCGCCGAGATTAGAGCCTCTGCCAATCTGGCCGCCAC
CAAGATGTCTGAGTGTGTGCTGGGCCAGAGCAAGAGAGTGGACTTTTGCGGCAAGGGCT
ACCACCTGATGAGCTTCCCTCAGTCTGCCCCTCACGGCGTGGTGTTTCTGCACGTGACAT
ACGTGCCCGCTCAAGAGAAGAATTTCACCACCGCTCCAGCCATCTGCCACGACGGCAAA
GCCCACTTTCCTAGAGAAGGCGTGTTCGTGTCCAACGGCACCCATTGGTTCGTGACCCA
GCGGAACTTCTACGAGCCCCAGATCATCACCACCGACAACACCTTCGTGTCTGGCAACTG
CGACGTCGTGATCGGCATTGTGAACAATACCGTGTACGACCCTCTGCAGCCCGAGCTGG
ACAGCTTCAAAGAGGAACTGGATAAGTACTTTAAGAACCACACAAGCCCCGACGTGGAC
CTGGGCGATATCAGCGGAATCAATGCCAGCGTCGTGAACATCCAGAAAGAGATCGACCG
GCTGAACGAGGTGGCCAAGAATCTGAACGAGAGCCTGATCGACCTGCAAGAACTGGGG
AAGTACGAGCAGTACATCAAGTGGCCCTGGTACATCTGGCTGGGCTTTATCGCCGGACT
GATTGCCATCGTGATGGTCACAATCATGCTGTGTTGCATGACCAGCTGCTGTAGCTGCCT
GAAGGGCTGTTGTAGCTGTGGCAGCTGCTGCAAGTTCGACGAGGACGATTCTGAGCCCG
TGCTGAAGGGCGTGAAACTGCACTACACC
SEQ ID NO: 2 (nucleic acid sequence for SARS-COV-2 S1 protein, codon
optimized for humans)
ATGTTCGTGTTTCTGGTGCTGCTGCCTCTGGTGTCCAGCCAGTGTGTGAACCTGACCACC
AGAACACAGCTGCCTCCAGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCCACGTGTCCGGCACCAATGGCACCAAGAGATTCGA
CAACCCCGTGCTGCCCTTCAACGACGGGGTGTACTTTGCCAGCACCGAGAAGTCCAACA
TCATCAGAGGCTGGATCTTCGGCACCACACTGGACAGCAAGACCCAGAGCCTGCTGATC
GTGAACAACGCCACCAACGTGGTCATCAAAGTGTGCGAGTTCCAGTTCTGCAACGACCC
CTTCCTGGGCGTCTACTACCACAAGAACAACAAGAGCTGGATGGAAAGCGAGTTCCGGG
TGTACAGCAGCGCCAACAACTGCACCTTCGAGTACGTGTCCCAGCCTTTCCTGATGGACC
TGGAAGGCAAGCAGGGCAACTTCAAGAACCTGCGCGAGTTCGTGTTCAAGAACATCGAC
GGCTACTTCAAGATCTACAGCAAGCACACCCCTATCAACCTCGTGCGGGATCTGCCTCAG
GGCTTCTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGCGGATG
GACAGCTGGTGCCGCCGCTTACTATGTGGGCTACCTGCAGCCTAGAACCTTCCTGCTGA
AGTACAACGAGAACGGCACCATCACCGACGCCGTGGATTGTGCTCTGGATCCTCTGAGC
GAGACAAAGTGCACCCTGAAGTCCTTCACCGTGGAAAAGGGCATCTACCAGACCAGCAA
CTTCCGGGTGCAGCCCACCGAATCCATCGTGCGGTTCCCCAATATCACCAATCTGTGCCC
CTTCGGCGAGGTGTTCAATGCCACCAGATTCGCCTCTGTGTACGCCTGGAACCGGAAGC
GGATCAGCAATTGCGTGGCCGACTACTCCGTGCTGTACAACTCCGCCAGCTTCAGCACCT
TCAAGTGCTACGGCGTGTCCCCTACCAAGCTGAACGACCTGTGCTTCACAAACGTGTACG
CCGACAGCTTCGTGATCCGGGGAGATGAAGTGCGGCAGATTGCCCCTGGACAGACAGG
CAAGATCGCCGACTACAACTACAAGCTGCCCGACGACTTCACCGGCTGTGTGATTGCCT
GGAACAGCAACAACCTGGACTCCAAAGTCGGCGGCAACTACAATTACCTGTACCGGCTG
TTCCGGAAGTCCAATCTGAAGCCCTTCGAGCGGGACATCTCCACCGAGATCTATCAGGC
CGGCAGCACCCCTTGTAACGGCGTGGAAGGCTTCAACTGCTACTTCCCACTGCAGTCCTA
CGGCTTTCAGCCCACAAATGGCGTGGGCTATCAGCCCTACAGAGTGGTGGTGCTGAGCT
TCGAACTGCTGCATGCCCCTGCCACAGTGTGCGGCCCTAAGAAAAGCACCAATCTCGTG
AAGAACAAATGCGTGAACTTCAACTTCAACGGCCTGACCGGCACCGGCGTGCTGACAGA
GAGCAACAAGAAGTTCCTGCCATTCCAGCAGTTTGGCCGGGATATCGCCGATACCACAG
ACGCCGTTAGAGATCCCCAGACACTGGAAATCCTGGACATCACCCCTTGCAGCTTCGGC
GGAGTGTCTGTGATCACCCCTGGCACCAACACCAGCAATCAGGTGGCAGTGCTGTACCA
GGACGTGAACTGTACCGAAGTGCCCGTGGCCATTCACGCCGATCAGCTGACACCTACAT
GGCGGGTGTACTCCACCGGCAGCAATGTGTTTCAGACCAGAGCCGGCTGTCTGATCGGA
GCCGAGCACGTGAACAATAGCTACGAGTGCGACATCCCCATCGGCGCTGGCATCTGTGC
CAGCTACCAGACACAGACAAACAGCCCCAGACGGGCCAGA
SEQ ID NO: 3 (nucleic acid sequence for SARS-COV-2 S2 protein, codon
optimized for humans)
TCTGTGGCCAGCCAGAGCATCATTGCCTACACAATGTCTCTGGGCGCCGAGAACAGCGT
GGCCTACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGCGGCG
ATTCCACCGAGTGCTCCAACCTGCTGCTGCAGTACGGCAGCTTCTGCACCCAGCTGAATA
GAGCCCTGACAGGGATCGCCGTGGAACAGGACAAGAACACCCAAGAGGTGTTCGCCCA
AGTGAAGCAGATCTACAAGACCCCTCCTATCAAGGACTTCGGCGGCTTCAATTTCAGCCA
GATTCTGCCCGATCCTAGCAAGCCCAGCAAGCGGAGCTTCATCGAGGACCTGCTGTTCA
ACAAAGTGACACTGGCCGACGCCGGCTTCATCAAGCAGTATGGCGATTGTCTGGGCGAC
ATTGCCGCCAGGGATCTGATTTGCGCCCAGAAGTTTAACGGACTGACAGTGCTGCCTCCT
CTGCTGACCGATGAGATGATCGCCCAGTACACATCTGCCCTGCTGGCCGGCACAATCAC
AAGCGGCTGGACATTTGGAGCTGGCGCCGCTCTGCAGATCCCCTTTGCTATGCAGATGG
CCTACCGGTTCAACGGCATCGGAGTGACCCAGAATGTGCTGTACGAGAACCAGAAGCTG
ATCGCCAACCAGTTCAACAGCGCCATCGGCAAGATCCAGGACAGCCTGAGCAGCACAGC
AAGCGCCCTGGGAAAGCTGCAGGACGTGGTCAACCAGAATGCCCAGGCACTGAACACCC
TGGTCAAGCAGCTGTCCTCCAACTTCGGCGCCATCAGCTCTGTGCTGAACGATATCCTGA
GCAGACTGGACAAGGTGGAAGCCGAGGTGCAGATCGACAGACTGATCACCGGAAGGCT
GCAGTCCCTGCAGACCTACGTTACCCAGCAGCTGATCAGAGCCGCCGAGATTAGAGCCT
CTGCCAATCTGGCCGCCACCAAGATGTCTGAGTGTGTGCTGGGCCAGAGCAAGAGAGTG
GACTTTTGCGGCAAGGGCTACCACCTGATGAGCTTCCCTCAGTCTGCCCCTCACGGCGT
GGTGTTTCTGCACGTGACATACGTGCCCGCTCAAGAGAAGAATTTCACCACCGCTCCAGC
CATCTGCCACGACGGCAAAGCCCACTTTCCTAGAGAAGGCGTGTTCGTGTCCAACGGCA
CCCATTGGTTCGTGACCCAGCGGAACTTCTACGAGCCCCAGATCATCACCACCGACAACA
CCTTCGTGTCTGGCAACTGCGACGTCGTGATCGGCATTGTGAACAATACCGTGTACGACC
CTCTGCAGCCCGAGCTGGACAGCTTCAAAGAGGAACTGGATAAGTACTTTAAGAACCAC
ACAAGCCCCGACGTGGACCTGGGCGATATCAGCGGAATCAATGCCAGCGTCGTGAACAT
CCAGAAAGAGATCGACCGGCTGAACGAGGTGGCCAAGAATCTGAACGAGAGCCTGATC
GACCTGCAAGAACTGGGGAAGTACGAGCAGTACATCAAGTGGCCCTGGTACATCTGGCT
GGGCTTTATCGCCGGACTGATTGCCATCGTGATGGTCACAATCATGCTGTGTTGCATGAC
CAGCTGCTGTAGCTGCCTGAAGGGCTGTTGTAGCTGTGGCAGCTGCTGCAAGTTCGACG
AGGACGATTCTGAGCCCGTGCTGAAGGGCGTGAAACTGCACTACACC
SEQ ID NO: 4 (nucleic acid sequence for SARS-COV-2 SPIKE RBM motif)
AACAGCAACAACCTGGACTCCAAAGTCGGCGGCAACTACAATTACCTGTACCGGCTGTTC
CGGAAGTCCAATCTGAAGCCCTTCGAGCGGGACATCTCCACCGAGATCTATCAGGCCGG
CAGCACCCCTTGTAACGGCGTGGAAGGCTTCAACTGCTACTTCCCACTGCAGTCCTACGG
CTTTCAGCCCACAAATGGCGTGGGCTATCAGCCCTAC
SEQ ID NO: 5 (nucleic acid sequence for SARS-COV-2 SPIKE RBD domain)
CGGGTGCAGCCCACCGAATCCATCGTGCGGTTCCCCAATATCACCAATCTGTGCCCCTTC
GGCGAGGTGTTCAATGCCACCAGATTCGCCTCTGTGTACGCCTGGAACCGGAAGCGGAT
CAGCAATTGCGTGGCCGACTACTCCGTGCTGTACAACTCCGCCAGCTTCAGCACCTTCAA
GTGCTACGGCGTGTCCCCTACCAAGCTGAACGACCTGTGCTTCACAAACGTGTACGCCG
ACAGCTTCGTGATCCGGGGAGATGAAGTGCGGCAGATTGCCCCTGGACAGACAGGCAA
GATCGCCGACTACAACTACAAGCTGCCCGACGACTTCACCGGCTGTGTGATTGCCTGGA
ACAGCAACAACCTGGACTCCAAAGTCGGCGGCAACTACAATTACCTGTACCGGCTGTTCC
GGAAGTCCAATCTGAAGCCCTTCGAGCGGGACATCTCCACCGAGATCTATCAGGCCGGC
AGCACCCCTTGTAACGGCGTGGAAGGCTTCAACTGCTACTTCCCACTGCAGTCCTACGGC
TTTCAGCCCACAAATGGCGTGGGCTATCAGCCCTACAGAGTGGTGGTGCTGAGCTTCGA
ACTGCTGCATGCCCCTGCCACAGTGTGCGGCCCTAAGAAAAGCACCAATCTCGTGAAGA
ACAAATGCGTGAACTTC
SEQ ID NO: 6 (amino acid sequence for SARS-COV-2 SPIKE protein)
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTGVLTESNKKFLPF
QQFGRDIADTTDAVRDPQTLEILDITPCSFGGVSVITPGTNTSNQVAVLYQDVNCTEVPVAI
HADQLTPTWRVYSTGSNVFQTRAGCLIGAEHVNNSYECDIPIGAGICASYQTQTNSPRRAR
SVASQSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKIQDSL
SSTASALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRL
NEVAKNLNESLIDLQELGKYEQYIKWPWYIWLGFIAGLIAIVMVTIMLCCMTSCCSCLKGCC
SCGSCCKFDEDDSEPVLKGVKLHYT
SEQ ID NO: 7 (amino acid sequence for SARS-COV-2 S1 protein)
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTGVLTESNKKFLPF
QQFGRDIADTTDAVRDPQTLEILDITPCSFGGVSVITPGTNTSNQVAVLYQDVNCTEVPVAI
HADQLTPTWRVYSTGSNVFQTRAGCLIGAEHVNNSYECDIPIGAGICASYQTQTNSPRRAR
SEQ ID NO: 8 (amino acid sequence for SARS-COV-2 S2 protein)
SVASQSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKIQDSL
SSTASALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRL
NEVAKNLNESLIDLQELGKYEQYIKWPWYIWLGFIAGLIAIVMVTIMLCCMTSCCSCLKGCC
SCGSCCKFDEDDSEPVLKGVKLHYT
SEQ ID NO: 9 (amino acid sequence for SARS-COV-2 Spike RBM)
NSNNLDSKVGGNYNYLYRLFRKSNLKPFERDISTEIYQAGSTPCNGVEGFNCYFPLQSYGFQ
PTNGVGYQPY
SEQ ID NO: 10 (amino acid sequence for SARS-COV-2 Spike RBD)
RVQPTESIVRFPNITNLCPFGEVFNATRFASVYAWNRKRISNCVADYSVLYNSASFSTFKCYG
VSPTKLNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTNGVGY
QPYRVVVLSFELLHAPATVCGPKKSTNLVKNKCVNF
SEQ ID NO: 11 (nucleotide sequence for the Kozak sequence)
GCCACCATG
SEQ ID NO: 12 (nucleic acid sequence for SARS-COV-2 B.1.351 SPIKE
protein, codon optimized for humans):
ATGTTCGTGTTTCTGGTGCTGCTGCCTCTGGTGTCCAGCCAGTGCGTGAACTTCACCACC
AGAACACAGCTGCCTCCAGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCCACGTGTCCGGCACCAATGGCACCAAGAGATTCGC
CAATCCTGTGCTGCCCTTCAACGACGGGGTGTACTTTGCCAGCACCGAGAAGTCCAACAT
CATCAGAGGCTGGATCTTCGGCACCACACTGGACAGCAAGACCCAGAGCCTGCTGATCG
TGAACAACGCCACCAACGTGGTCATCAAAGTGTGCGAGTTCCAGTTCTGCAACGACCCCT
TCCTGGGCGTCTACTACCACAAGAACAACAAGAGCTGGATGGAAAGCGAGTTCCGGGTG
TACAGCAGCGCCAACAACTGCACCTTCGAGTACGTGTCCCAGCCTTTCCTGATGGACCTG
GAAGGCAAGCAGGGCAACTTCAAGAACCTGCGCGAGTTCGTGTTCAAGAACATCGACGG
CTACTTCAAGATCTACAGCAAGCACACCCCTATCAACCTCGTGCGGGGACTGCCTCAGG
GCTTTTCTGCTCTGGAACCCCTGGTGGATCTGCCCATCGGCATCAACATCACCCGGTTTC
AGACCCTGCACCGGTCCTATCTGACACCCGGCGATTCTTCTAGCGGATGGACAGCTGGC
GCCGCTGCCTACTATGTGGGATACCTGCAGCCTCGGACCTTCCTGCTGAAGTACAACGA
GAACGGCACCATCACCGACGCCGTGGATTGTGCTCTGGATCCTCTGAGCGAGACAAAGT
GCACCCTGAAGTCCTTCACCGTGGAAAAGGGCATCTACCAGACCAGCAACTTCCGGGTG
CAGCCCACCGAATCCATCGTGCGGTTCCCCAATATCACCAATCTGTGCCCCTTCGGCGAG
GTGTTCAATGCCACCAGATTCGCCTCTGTGTACGCCTGGAACCGGAAGCGGATCAGCAA
TTGCGTGGCCGACTACTCCGTGCTGTACAACTCCGCCAGCTTCAGCACCTTCAAGTGCTA
CGGCGTGTCCCCTACCAAGCTGAACGACCTGTGCTTCACAAACGTGTACGCCGACAGCT
TCGTGATCCGGGGAGATGAAGTGCGGCAGATTGCCCCTGGACAGACCGGCAATATCGCC
GACTACAACTACAAGCTGCCCGACGACTTCACCGGCTGTGTGATTGCCTGGAACAGCAA
CAACCTGGACTCCAAAGTCGGCGGCAACTACAATTACCTGTACCGGCTGTTCCGGAAGTC
CAATCTGAAGCCCTTCGAGCGGGACATCTCCACCGAGATCTATCAGGCCGGCAGCACCC
CTTGCAATGGCGTGAAGGGCTTTAACTGCTACTTCCCACTGCAGTCCTACGGCTTCCAGC
CAACATACGGCGTGGGCTACCAGCCTTACAGAGTGGTGGTGCTGAGCTTCGAGCTGCTG
CATGCTCCTGCCACAGTGTGCGGCCCTAAGAAAAGCACCAATCTCGTGAAGAACAAATG
CGTCAACTTCAATTTCAACGGCCTGACCGGCACCGGCGTGCTGACAGAGAGCAACAAGA
AGTTCCTGCCATTCCAGCAGTTCGGCCGGGACATTGCCGATACCACAGATGCTGTCAGA
GATCCCCAGACACTGGAAATCCTGGACATCACCCCATGCAGCTTCGGCGGAGTGTCTGT
GATCACCCCTGGCACCAACACCAGCAATCAGGTGGCAGTGCTGTACCAGGGCGTCAACT
GTACAGAGGTGCCAGTGGCCATTCACGCCGATCAGCTGACCCCTACTTGGCGGGTGTAC
TCCACAGGCAGCAATGTGTTCCAGACCAGAGCCGGCTGTCTGATCGGAGCCGAGCACGT
GAACAATAGCTACGAGTGCGACATCCCCATCGGCGCTGGCATCTGTGCCAGCTACCAGA
CACAGACAAACAGCCCCAGACGGGCCAGATCTGTGGCCAGCCAGAGCATCATTGCCTAC
ACAATGTCTCTGGGCGTCGAGAACAGCGTGGCCTACTCCAACAACTCTATCGCTATCCCC
ACCAATTTCACCATCAGCGTGACCACAGAGATCCTGCCTGTGTCCATGACCAAGACCAGC
GTGGACTGCACCATGTACATCTGCGGCGATAGCACCGAGTGCTCCAACCTGCTGCTGCA
GTACGGCAGCTTCTGCACCCAGCTGAATAGAGCCCTGACCGGAATCGCCGTGGAACAGG
ACAAGAACACCCAAGAGGTGTTCGCCCAAGTGAAGCAGATCTACAAGACCCCTCCTATCA
AGGACTTCGGCGGCTTCAACTTCAGCCAGATTCTGCCCGATCCTAGCAAGCCCAGCAAG
CGGAGCTTCATCGAGGACCTGCTGTTCAACAAAGTGACACTGGCCGACGCCGGCTTCAT
CAAGCAGTATGGCGATTGTCTGGGCGACATTGCAGCCCGGGATCTGATTTGCGCCCAGA
AGTTTAACGGACTGACCGTGCTGCCTCCTCTGCTGACCGATGAGATGATCGCCCAGTACA
CATCTGCCCTGCTGGCCGGCACAATCACAAGCGGCTGGACATTTGGAGCTGGCGCTGCC
CTGCAGATCCCCTTTGCTATGCAGATGGCCTACCGGTTCAACGGCATCGGAGTGACCCA
GAATGTGCTGTACGAGAACCAGAAGCTGATCGCCAACCAGTTCAACAGCGCCATCGGCA
AGATCCAGGACAGCCTGAGCAGCACAGCCAGCGCTCTGGGAAAACTGCAGGACGTGGT
CAACCAGAACGCCCAGGCTCTGAATACCCTGGTCAAGCAGCTGTCCTCCAACTTCGGCG
CCATCAGCTCTGTGCTGAACGATATCCTGAGCAGACTGGACAAGGTGGAAGCCGAGGTG
CAGATCGACAGACTGATCACCGGAAGGCTGCAGTCCCTGCAGACCTACGTTACCCAGCA
GCTGATCAGAGCCGCCGAGATTAGAGCCTCTGCCAATCTGGCCGCCACCAAGATGTCTG
AGTGTGTGCTGGGCCAGAGCAAGAGAGTGGACTTTTGCGGCAAGGGCTACCACCTGATG
AGCTTCCCTCAGTCTGCACCACACGGCGTGGTGTTTCTGCACGTGACATACGTGCCCGCT
CAAGAGAAGAACTTCACAACAGCCCCTGCCATCTGCCACGACGGCAAAGCCCACTTTCCT
AGAGAAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTCGTGA
TCGGCATTGTGAACAATACCGTGTACGACCCTCTGCAGCCCGAGCTGGACAGCTTCAAA
GAGGAACTGGATAAGTACTTTAAGAACCACACAAGCCCCGACGTGGACCTGGGCGATAT
CAGCGGAATCAATGCCAGCGTCGTGAACATCCAGAAAGAGATCGACCGGCTGAACGAGG
TGGCCAAGAATCTGAACGAGAGCCTGATCGACCTGCAAGAACTGGGGAAGTACGAGCAG
TACATCAAGTGGCCTTGGTACATCTGGCTGGGCTTTATCGCCGGACTGATTGCCATCGTG
ATGGTCACAATCATGCTGTGCTGTATGACCAGCTGCTGTAGCTGCCTGAAGGGCTGTTGC
AGCTGTGGCTCCTGCTGCAAGTTCGACGAGGACGATTCTGAGCCCGTGCTGAAGGGCGT
GAAACTGCACTACACC
SEQ ID NO: 13 (nucleic acid sequence for SARS-COV-2 B.1.351 S1 protein,
codon optimized for humans)
ATGTTCGTGTTTCTGGTGCTGCTGCCTCTGGTGTCCAGCCAGTGCGTGAACTTCACCACC
AGAACACAGCTGCCTCCAGCCTACACCAACAGCTTTACCAGAGGCGTGTACTACCCCGAC
AAGGTGTTCAGATCCAGCGTGCTGCACTCTACCCAGGACCTGTTCCTGCCTTTCTTCAGC
AACGTGACCTGGTTCCACGCCATCCACGTGTCCGGCACCAATGGCACCAAGAGATTCGC
CAATCCTGTGCTGCCCTTCAACGACGGGGTGTACTTTGCCAGCACCGAGAAGTCCAACAT
CATCAGAGGCTGGATCTTCGGCACCACACTGGACAGCAAGACCCAGAGCCTGCTGATCG
TGAACAACGCCACCAACGTGGTCATCAAAGTGTGCGAGTTCCAGTTCTGCAACGACCCCT
TCCTGGGCGTCTACTACCACAAGAACAACAAGAGCTGGATGGAAAGCGAGTTCCGGGTG
TACAGCAGCGCCAACAACTGCACCTTCGAGTACGTGTCCCAGCCTTTCCTGATGGACCTG
GAAGGCAAGCAGGGCAACTTCAAGAACCTGCGCGAGTTCGTGTTCAAGAACATCGACGG
CTACTTCAAGATCTACAGCAAGCACACCCCTATCAACCTCGTGCGGGGACTGCCTCAGG
GCTTTTCTGCTCTGGAACCCCTGGTGGATCTGCCCATCGGCATCAACATCACCCGGTTTC
AGACCCTGCACCGGTCCTATCTGACACCCGGCGATTCTTCTAGCGGATGGACAGCTGGC
GCCGCTGCCTACTATGTGGGATACCTGCAGCCTCGGACCTTCCTGCTGAAGTACAACGA
GAACGGCACCATCACCGACGCCGTGGATTGTGCTCTGGATCCTCTGAGCGAGACAAAGT
GCACCCTGAAGTCCTTCACCGTGGAAAAGGGCATCTACCAGACCAGCAACTTCCGGGTG
CAGCCCACCGAATCCATCGTGCGGTTCCCCAATATCACCAATCTGTGCCCCTTCGGCGAG
GTGTTCAATGCCACCAGATTCGCCTCTGTGTACGCCTGGAACCGGAAGCGGATCAGCAA
TTGCGTGGCCGACTACTCCGTGCTGTACAACTCCGCCAGCTTCAGCACCTTCAAGTGCTA
CGGCGTGTCCCCTACCAAGCTGAACGACCTGTGCTTCACAAACGTGTACGCCGACAGCT
TCGTGATCCGGGGAGATGAAGTGCGGCAGATTGCCCCTGGACAGACCGGCAATATCGCC
GACTACAACTACAAGCTGCCCGACGACTTCACCGGCTGTGTGATTGCCTGGAACAGCAA
CAACCTGGACTCCAAAGTCGGCGGCAACTACAATTACCTGTACCGGCTGTTCCGGAAGTC
CAATCTGAAGCCCTTCGAGCGGGACATCTCCACCGAGATCTATCAGGCCGGCAGCACCC
CTTGCAATGGCGTGAAGGGCTTTAACTGCTACTTCCCACTGCAGTCCTACGGCTTCCAGC
CAACATACGGCGTGGGCTACCAGCCTTACAGAGTGGTGGTGCTGAGCTTCGAGCTGCTG
CATGCTCCTGCCACAGTGTGCGGCCCTAAGAAAAGCACCAATCTCGTGAAGAACAAATG
CGTCAACTTCAATTTCAACGGCCTGACCGGCACCGGCGTGCTGACAGAGAGCAACAAGA
AGTTCCTGCCATTCCAGCAGTTCGGCCGGGACATTGCCGATACCACAGATGCTGTCAGA
GATCCCCAGACACTGGAAATCCTGGACATCACCCCATGCAGCTTCGGCGGAGTGTCTGT
GATCACCCCTGGCACCAACACCAGCAATCAGGTGGCAGTGCTGTACCAGGGCGTCAACT
GTACAGAGGTGCCAGTGGCCATTCACGCCGATCAGCTGACCCCTACTTGGCGGGTGTAC
TCCACAGGCAGCAATGTGTTCCAGACCAGAGCCGGCTGTCTGATCGGAGCCGAGCACGT
GAACAATAGCTACGAGTGCGACATCCCCATCGGCGCTGGCATCTGTGCCAGCTACCAGA
CACAGACAAACAGCCCCAGACGGGCCAGA
SEQ ID NO: 14 (nucleic acid sequence for SARS-COV-2 B.1.351 S2 protein,
codon optimized for humans)
TCTGTGGCCAGCCAGAGCATCATTGCCTACACAATGTCTCTGGGCGTCGAGAACAGCGT
GGCCTACTCCAACAACTCTATCGCTATCCCCACCAATTTCACCATCAGCGTGACCACAGA
GATCCTGCCTGTGTCCATGACCAAGACCAGCGTGGACTGCACCATGTACATCTGCGGCG
ATAGCACCGAGTGCTCCAACCTGCTGCTGCAGTACGGCAGCTTCTGCACCCAGCTGAAT
AGAGCCCTGACCGGAATCGCCGTGGAACAGGACAAGAACACCCAAGAGGTGTTCGCCCA
AGTGAAGCAGATCTACAAGACCCCTCCTATCAAGGACTTCGGCGGCTTCAACTTCAGCCA
GATTCTGCCCGATCCTAGCAAGCCCAGCAAGCGGAGCTTCATCGAGGACCTGCTGTTCA
ACAAAGTGACACTGGCCGACGCCGGCTTCATCAAGCAGTATGGCGATTGTCTGGGCGAC
ATTGCAGCCCGGGATCTGATTTGCGCCCAGAAGTTTAACGGACTGACCGTGCTGCCTCCT
CTGCTGACCGATGAGATGATCGCCCAGTACACATCTGCCCTGCTGGCCGGCACAATCAC
AAGCGGCTGGACATTTGGAGCTGGCGCTGCCCTGCAGATCCCCTTTGCTATGCAGATGG
CCTACCGGTTCAACGGCATCGGAGTGACCCAGAATGTGCTGTACGAGAACCAGAAGCTG
ATCGCCAACCAGTTCAACAGCGCCATCGGCAAGATCCAGGACAGCCTGAGCAGCACAGC
CAGCGCTCTGGGAAAACTGCAGGACGTGGTCAACCAGAACGCCCAGGCTCTGAATACCC
TGGTCAAGCAGCTGTCCTCCAACTTCGGCGCCATCAGCTCTGTGCTGAACGATATCCTGA
GCAGACTGGACAAGGTGGAAGCCGAGGTGCAGATCGACAGACTGATCACCGGAAGGCT
GCAGTCCCTGCAGACCTACGTTACCCAGCAGCTGATCAGAGCCGCCGAGATTAGAGCCT
CTGCCAATCTGGCCGCCACCAAGATGTCTGAGTGTGTGCTGGGCCAGAGCAAGAGAGTG
GACTTTTGCGGCAAGGGCTACCACCTGATGAGCTTCCCTCAGTCTGCACCACACGGCGT
GGTGTTTCTGCACGTGACATACGTGCCCGCTCAAGAGAAGAACTTCACAACAGCCCCTGC
CATCTGCCACGACGGCAAAGCCCACTTTCCTAGAGAAGGCGTGTTCGTGTCCAACGGCA
CCCATTGGTTCGTGACCCAGCGGAACTTCTACGAGCCCCAGATCATCACCACCGACAACA
CCTTCGTGTCTGGCAACTGCGACGTCGTGATCGGCATTGTGAACAATACCGTGTACGACC
CTCTGCAGCCCGAGCTGGACAGCTTCAAAGAGGAACTGGATAAGTACTTTAAGAACCAC
ACAAGCCCCGACGTGGACCTGGGCGATATCAGCGGAATCAATGCCAGCGTCGTGAACAT
CCAGAAAGAGATCGACCGGCTGAACGAGGTGGCCAAGAATCTGAACGAGAGCCTGATC
GACCTGCAAGAACTGGGGAAGTACGAGCAGTACATCAAGTGGCCTTGGTACATCTGGCT
GGGCTTTATCGCCGGACTGATTGCCATCGTGATGGTCACAATCATGCTGTGCTGTATGAC
CAGCTGCTGTAGCTGCCTGAAGGGCTGTTGCAGCTGTGGCTCCTGCTGCAAGTTCGACG
AGGACGATTCTGAGCCCGTGCTGAAGGGCGTGAAACTGCACTACACC
SEQ ID NO: 15 (nucleotide sequence for SARS-COV-2 B.1.351 SPIKE RBM
motif, codon optimized for humans)
AACAGCAACAACCTGGACTCCAAAGTCGGCGGCAACTACAATTACCTGTACCGGCTGTTC
CGGAAGTCCAATCTGAAGCCCTTCGAGCGGGACATCTCCACCGAGATCTATCAGGCCGG
CAGCACCCCTTGCAATGGCGTGAAGGGCTTTAACTGCTACTTCCCACTGCAGTCCTACGG
CTTCCAGCCAACATACGGCGTGGGCTACCAGCCTTAC
SEQ ID NO: 16 (nucleotide sequence for SARS-COV-2 B.1.351 SPIKE RBD
domain, codon optimized for humans)
CGGGTGCAGCCCACCGAATCCATCGTGCGGTTCCCCAATATCACCAATCTGTGCCCCTTC
GGCGAGGTGTTCAATGCCACCAGATTCGCCTCTGTGTACGCCTGGAACCGGAAGCGGAT
CAGCAATTGCGTGGCCGACTACTCCGTGCTGTACAACTCCGCCAGCTTCAGCACCTTCAA
GTGCTACGGCGTGTCCCCTACCAAGCTGAACGACCTGTGCTTCACAAACGTGTACGCCG
ACAGCTTCGTGATCCGGGGAGATGAAGTGCGGCAGATTGCCCCTGGACAGACCGGCAAT
ATCGCCGACTACAACTACAAGCTGCCCGACGACTTCACCGGCTGTGTGATTGCCTGGAA
CAGCAACAACCTGGACTCCAAAGTCGGCGGCAACTACAATTACCTGTACCGGCTGTTCCG
GAAGTCCAATCTGAAGCCCTTCGAGCGGGACATCTCCACCGAGATCTATCAGGCCGGCA
GCACCCCTTGCAATGGCGTGAAGGGCTTTAACTGCTACTTCCCACTGCAGTCCTACGGCT
TCCAGCCAACATACGGCGTGGGCTACCAGCCTTACAGAGTGGTGGTGCTGAGCTTCGAG
CTGCTGCATGCTCCTGCCACAGTGTGCGGCCCTAAGAAAAGCACCAATCTCGTGAAGAA
CAAATGCGTCAACTTC
SEQ ID NO: 17 (amino acid sequence for SARS-COV-2 B.1.351 SPIKE
protein)
MFVFLVLLPLVSSQCVNFTTRTQLPPAYTNSFTRGVYYPDKVFRSSVLHSTQDLFLPFFSNVT
WFHAIHVSGTNGTKRFANPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNF
KNLREFVFKNIDGYFKIYSKHTPINLVRGLPQGFSALEPLVDLPIGINITRFQTLHRSYLTPGDS
SSGWTAGAAAYYVGYLQPRTFLLKYNENGTITDAVDCALDPLSETKCTLKSFTVEKGIYQTS
NFRVQPTESIVRFPNITNLCPFGEVFNATRFASVYAWNRKRISNCVADYSVLYNSASFSTFKC
YGVSPTKLNDLCFTNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYFPLQSYGFQPTYGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQTQTNSPRRARSVAS
QSIIAYTMSLGVENSVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPLLTDEMIAQYTSALLAG
TITSGWTFGAGAALQIPFAMQMAYRFNGIGVTQNVLYENQKLIANQFNSAIGKIQDSLSSTA
SALGKLQDVVNQNAQALNTLVKQLSSNFGAISSVLNDILSRLDKVEAEVQIDRLITGRLQSL
QTYVTQQLIRAAEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQIITTDNTFVSGNCD
VVIGIVNNTVYDPLQPELDSFKEELDKYFKNHTSPDVDLGDISGINASVVNIQKEIDRLNEVA
KNLNESLIDLQELGKYEQYIKWPWYIWLGFIAGLIAIVMVTIMLCCMTSCCSCLKGCCSCGS
CCKFDEDDSEPVLKGVKLHYT
SEQ ID NO: 18 (amino acid sequence for SARS-COV-2 B.1.351 S1 protein)
MFVFLVLLPLVSSQCVNFTTRTQLPPAYTNSFTRGVYYPDKVFRSSVLHSTQDLFLPFFSNVT
WFHAIHVSGTNGTKRFANPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNF
KNLREFVFKNIDGYFKIYSKHTPINLVRGLPQGFSALEPLVDLPIGINITRFQTLHRSYLTPGDS
SSGWTAGAAAYYVGYLQPRTFLLKYNENGTITDAVDCALDPLSETKCTLKSFTVEKGIYQTS
NFRVQPTESIVRFPNITNLCPFGEVFNATRFASVYAWNRKRISNCVADYSVLYNSASFSTFKC
YGVSPTKLNDLCFTNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYFPLQSYGFQPTYGV
GYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQTQTNSPRRAR
SEQ ID NO: 19 (amino acid sequence for SARS-COV-2 B.1.351 S2 protein)
SVASQSIIAYTMSLGVENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKIQDSL
SSTASALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRL
NEVAKNLNESLIDLQELGKYEQYIKWPWYIWLGFIAGLIAIVMVTIMLCCMTSCCSCLKGCC
SCGSCCKFDEDDSEPVLKGVKLHYT
SEQ ID NO: 20 (amino acid sequence for SARS-COV-2 B.1.351 SPIKE RBM)
NSNNLDSKVGGNYNYLYRLFRKSNLKPFERDISTEIYQAGSTPCNGVKGFNCYFPLQSYGFQ
PTYGVGYQPY
SEQ ID NO: 21 (amino acid sequence for SARS-COV-2 B.1.351 SPIKE RBD)
RVQPTESIVRFPNITNLCPFGEVFNATRFASVYAWNRKRISNCVADYSVLYNSASFSTFKCYG
VSPTKLNDLCFTNVYADSFVIRGDEVRQIAPGQTGNIADYNYKLPDDFTGCVIAWNSNNLDS
KVGGNYNYLYRLFRKSNLKPFERDISTEIYQAGSTPCNGVKGFNCYFPLQSYGFQPTYGVGY
QPYRVVVLSFELLHAPATVCGPKKSTNLVKNKCVNF

1. A DNA vaccine comprising a DNA construct
a. with the nucleic acid sequence SEQ ID NO: 1 encoding a modified SPIKE protein that originates from the corona virus SARS-COV-2 and/or the nucleic acid sequence SEQ ID NO: 12 encoding a modified SPIKE protein that originates from SARS-COV-2 variant B.1.351 or a fragment thereof having at least 80% sequence identity to SEQ ID NO: 1 or 12, preferably 90%, more preferably 95% identity to SEQ ID NO: 1 or 12;
b. with the nucleic acid sequence SEQ ID NO: 2 encoding a modified SPIKE S1 protein that originates from the corona virus SARS-COV-2 and/or the nucleic acid sequence SEQ ID NO: 13 encoding a modified SPIKE S1 protein that originates from SARS-COV-2 variant B.1.351 or a fragment thereof having at least 80% sequence identity to SEQ ID NO: 2 or 13, preferably 90%, more preferably 95% identity to SEQ ID NO: 2 or 13;
c. with the nucleic acid sequence SEQ ID NO: 3 encoding a modified SPIKE S2 protein that originates from the corona virus SARS-COV-2 and/or the nucleic acid sequence SEQ ID NO: 14 encoding a modified SPIKE S2 protein that originates from SARS-COV-2 variant B.1.351 or a fragment thereof having at least 80% sequence identity to SEQ ID NO: 3 or 14, preferably 90%, more preferably 95% identity to SEQ ID NO: 3 or 14;
d. with the nucleic acid sequence SEQ ID NO: 4 encoding a modified RBM protein that originates from SARS-COV-2 and/or the nucleic acid sequence SEQ ID NO: 15 encoding a modified RBM protein that originates from SARS-COV-2 variant B.1.351 or a fragment thereof having at least 80% sequence identity to SEQ ID NO: 4 or 15, preferably 90%, more preferably 95% identity to SEQ ID NO: 4 or 15;
e. with the nucleic acid sequence SEQ ID NO: 5 encoding a modified RBD protein that originates from SARS-COV-2 and/or the nucleic acid sequence SEQ ID NO: 16 encoding a modified RBD protein that originates from SARS-COV-2 variant B.1.351 or a fragment thereof having at least 80% sequence identity to SEQ ID NO: 5 or 16, preferably 90%, more preferably 95% identity to SEQ ID NO: 5 or 16; or
f. encoding an amino acid sequence according to SEQ ID NO: 6 encoding a modified SPIKE protein that originates from the corona virus SARS-COV-2 and/or a DNA construct encoding an amino acid sequence according to SEQ ID NO: 17 encoding a modified SPIKE protein that originates from the corona virus SARS-COV-2 variant B.1.351 or a fragment thereof having at least 80% sequence identity to SEQ ID NO: 6 or 17, preferably 90%, more preferably 95% identity to SEQ ID NO: 6 or 17.

2.-6. (canceled)

7. The DNA vaccine according to claim 1, wherein the nucleic acid sequences according to SEQ ID: 1-6 stem from the Wuhan-Hu-1 (MN908947) strain.

8. The DNA vaccine according to claim 1, wherein said protein comprises an epitope that binds MHC class I protein or MHC class II protein.

9. (canceled)

10. The DNA vaccine according to claim 1, wherein the DNA construct is inserted into a vector.

11. The DNA vaccine according to claim 10, wherein the vector is a eukaryotic expression vector comprising the DNA construct operatively linked to
(A) a promotor; and
(B) Optionally, additionally regulatory sequences that regulate expression of said DNA construct.

12. (canceled)

13. The DNA vaccine according to claim 1, wherein the expression vector comprises the following regulatory sequences; a CMV promoter, the DNA construct, a Kozak translation initiation sequence, a polyadenylation signal, origin of replication and a selection marker.

14. The DNA vaccine according to claim 13, wherein the selection marker is antibiotic free RNA-OUT antisense RNA selection.

15. The DNA vaccine according to claim 13, wherein the Kozak translation initiation sequence is SEQ ID NO: 11.

16. The DNA vaccine according to claim 13, wherein the expression vector is the NTC8685-eRNA41H.

17. A method of preventing, vaccinating and/or immunizing a subject against infections and/or disease caused by SARS-COV-2 comprising administering the DNA vaccine according to claim 1.

18. The method according to claim 17, wherein the vaccine is administered to the subject by intradermal, intravenous, intramuscular or subcutaneous injection.

19. The method according to claim 17, wherein the vaccine is administered to said subject by needle injection or a needle free injection.

20. The method according to claim 19, wherein the needle free injection is a needle free jet injection or uses a stream of fluid to penetrate the skin.

21. (canceled)

22. The method according to claim 17, wherein the subject is selected from the group consisting of humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds, preferably the subject is human.

23. (canceled)

24. The method according to claim 17, wherein the vaccine is administered as
a. a single dose;
b. a first dose followed by a booster dose two weeks later, such as three weeks later; or
c. a first dose followed by two booster doses two weeks apart, such as three weeks apart.

25.-26. (canceled)

27. The method according to claim 24,
a. wherein the first dose is administered as one or more doses comprising one or more DNA constructs with anyone of the nucleic acid sequences SEQ ID NO: 1-5 or any sequences encoding the amino acid sequences according to SEQ ID NO: 6-10 and/or one or more DNA constructs with anyone of the nucleic acid sequences SEQ ID NO: 12-16 or any sequences encoding the amino acid sequences according to SEQ ID NO: 17-21;
b. wherein the booster dose is administered as one or more doses comprising the same or different DNA constructs with anyone of the nucleic acid sequences SEQ ID NO: 1-5 or any sequences encoding the amino acid sequences according to SEQ ID NO: 6-10 and/or with anyone of the nucleic acid sequences SEQ ID NO: 12-16 or any sequences encoding the amino acid sequences according to SEQ ID NO: 17-21;
c. comprising a DNA construct with anyone of the nucleic acid sequences SEQ ID NO: 1-5 or any sequences encoding the amino acid sequences according to SEQ ID NO: 6-10 is administered in a first dose followed by one booster dose two weeks later, such as two booster doses two weeks apart, wherein the booster dose comprises the DNA vaccine comprising a DNA construct with anyone of the nucleic acid sequences SEQ ID NO: 12-16 or any sequences encoding the amino acid sequences according to SEQ ID NO: 17-21;
d. comprising a DNA construct with anyone of the SEQ ID NO: 12-16 or any sequences encoding the amino acid sequences according to SEQ ID NO: 17-21 is administered in a first dose followed by one booster dose two weeks later, such as two booster doses two weeks apart, wherein the booster dose comprises the DNA vaccine comprising DNA construct with anyone of the SEQ ID NO: 1-5 or any sequences encoding the amino acid sequences according to SEQ ID NO: 6-10.

28.-30. (canceled)

31. The method according to claim 17, wherein the vaccine is administered in a dose of 0.5 mg, such as 1 mg, such as 2 mg, such as 3 mg, such as 4 mg, such as 5 mg.

32. The DNA vaccine according to claim 1 further comprising one or more adjuvants.

33. A pharmaceutical composition comprising the DNA construct inserted into the vector according to claim 1, preferably the composition is effective against any genotypic variant of SARS-COV-2.

34.-35. (canceled)

36. A method for inducing a protective immune response to SARS-CoV-2 comprising; administering said composition according to claim 31 to a subject by intradermal, intravenous, intramuscular or subcutaneous injection or by inhalation.


SEQUENCE LISTING (21 sequences)

SEQ ID NO: 1 - DNA, 3819 bp, Artificial Sequence
  sequence encoding modified SPIKE protein
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacc

SEQ ID NO: 2 - DNA, 2055 bp, Artificial Sequence
  sequence encoding a modified SPIKE S1 protein
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccaga

SEQ ID NO: 3 - DNA, 1764 bp, Artificial Sequence
  sequence encoding a modified SPIKE S2 protein
tctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacc

SEQ ID NO: 4 - DNA, 216 bp, Artificial Sequence
  sequence encoding a modified receptor binding motif (RBM) protein
aacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctac

SEQ ID NO: 5 - DNA, 669 bp, Artificial Sequence
  sequence encoding a modified receptor binding domain (RBD)
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttc

SEQ ID NO: 6 - protein, 1273 aa, Artificial Sequence
  sequence encoding a modified SPIKE protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 685 aa, Artificial Sequence
  sequence encoding a modified SPIKE S1 protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 8 - protein, 588 aa, Artificial Sequence
  sequence encoding a modified SPIKE S2 protein
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 72 aa, Artificial Sequence
  sequence encoding a modified RBM protein
NSNNLDSKVGGNYNYLYRLFRKSNLKPFERDISTEIYQAGSTPCNGVEGFNCYFPLQSYGFQPTNGVGYQPY

SEQ ID NO: 10 - protein, 223 aa, Artificial Sequence
  sequence encoding a modified RBM protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 11 - DNA, 9 bp, Artificial Sequence
  Kozak sequence
gccaccatg

SEQ ID NO: 12 - DNA, 3810 bp, Artificial Sequence
  sequence encoding a modified SPIKE protein
atgttcgtgtttctggtgctgctgcctctggtgtccagccagtgcgtgaacttcaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaatcctgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggactgcctcagggcttttctgctctggaacccctggtggatctgcccatcggcatcaacatcacccggtttcagaccctgcaccggtcctatctgacacccggcgattcttctagcggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgcaatggcgtgaagggctttaactgctacttcccactgcagtcctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtcaacttcaatttcaacggcctgaccggcaccggcgtgctgacagagagcaacaagaagttcctgccattccagcagttcggccgggacattgccgataccacagatgctgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgttccagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgtcgagaacagcgtggcctactccaacaactctatcgctatccccaccaatttcaccatcagcgtgaccacagagatcctgcctgtgtccatgaccaagaccagcgtggactgcaccatgtacatctgcggcgatagcaccgagtgctccaacctgctgctgcagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtatggcgattgtctgggcgacattgcagcccgggatctgatttgcgcccagaagtttaacggactgaccgtgctgcctcctctgctgaccgatgagatgatcgcccagtacacatctgccctgctggccggcacaatcacaagcggctggacatttggagctggcgctgccctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagccagcgctctgggaaaactgcaggacgtggtcaaccagaacgcccaggctctgaataccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaacttcacaacagcccctgccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgcagctgtggctcctgctgcaagttcgacgaggacgattctgagcccgtgctgaagggcgtgaaactgcactacacc

SEQ ID NO: 13 - DNA, 2046 bp, Artificial Sequence
  sequence encoding a modified SPIKE S1 protein
atgttcgtgtttctggtgctgctgcctctggtgtccagccagtgcgtgaacttcaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaatcctgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggactgcctcagggcttttctgctctggaacccctggtggatctgcccatcggcatcaacatcacccggtttcagaccctgcaccggtcctatctgacacccggcgattcttctagcggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgcaatggcgtgaagggctttaactgctacttcccactgcagtcctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtcaacttcaatttcaacggcctgaccggcaccggcgtgctgacagagagcaacaagaagttcctgccattccagcagttcggccgggacattgccgataccacagatgctgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgttccagaccagagccggctgtctgatcggagccgagcacgtgaacaatagctacgagtgcgacatccccatcggcgctggcatctgtgccagctaccagacacagacaaacagccccagacgggccaga

SEQ ID NO: 14 - DNA, 1764 bp, Artificial Sequence
  sequence encoding a modified SPIKE S2 protein
tctgtggccagccagagcatcattgcctacacaatgtctctgggcgtcgagaacagcgtggcctactccaacaactctatcgctatccccaccaatttcaccatcagcgtgaccacagagatcctgcctgtgtccatgaccaagaccagcgtggactgcaccatgtacatctgcggcgatagcaccgagtgctccaacctgctgctgcagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtatggcgattgtctgggcgacattgcagcccgggatctgatttgcgcccagaagtttaacggactgaccgtgctgcctcctctgctgaccgatgagatgatcgcccagtacacatctgccctgctggccggcacaatcacaagcggctggacatttggagctggcgctgccctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagccagcgctctgggaaaactgcaggacgtggtcaaccagaacgcccaggctctgaataccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaacttcacaacagcccctgccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgcagctgtggctcctgctgcaagttcgacgaggacgattctgagcccgtgctgaagggcgtgaaactgcactacacc

SEQ ID NO: 15 - DNA, 216 bp, Artificial Sequence
  sequence encoding a modified receptor binding motif (RBM)
aacagcaacaacctggactccaaagtcggcggcaactacaattacctgtaccggctgttccggaagtccaatctgaagcccttcgagcgggacatctccaccgagatctatcaggccggcagcaccccttgcaatggcgtgaagggctttaactgctacttcccactgcagtcctacggcttccagccaacatacggcgtgggctaccagccttac

SEQ ID NO: 16 - DNA, 669 bp, Artificial Sequence
  sequence encoding a modified receptor binding domain (RBD)
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgcaatggcgtgaagggctttaactgctacttcccactgcagtcctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtcaacttc

SEQ ID NO: 17 - protein, 1270 aa, Artificial Sequence
  sequence encoding a modified SPIKE protein
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 682 aa, Artificial Sequence
  sequence encoding a modified SPIKE S1 protein
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAR

SEQ ID NO: 19 - protein, 588 aa, Artificial Sequence
  sequence encoding modified SPIKE S2 protein
SVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 72 aa, Artificial Sequence
  sequence encoding a modified RBM protein
NSNNLDSKVGGNYNYLYRLFRKSNLKPFERDISTEIYQAGSTPCNGVKGFNCYFPLQSYGFQPTYGVGYQPY

SEQ ID NO: 21 - protein, 223 aa, Artificial Sequence
  sequence encoding a modified RBD protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF